SEQUENCE LISTING (93 sequences)

SEQ ID NO: 1 - DNA, 20 bp, Artificial Sequence
  synthetic
gaggcagggacagagttcag

SEQ ID NO: 2 - DNA, 24 bp, Artificial Sequence
  synthetic
agaagaacaggcagatgctaagag

SEQ ID NO: 3 - DNA, 19 bp, Artificial Sequence
  synthetic
tgaggtgcactctctatta

SEQ ID NO: 4 - DNA, 20 bp, Artificial Sequence
  synthetic
tgaggtgcactttctattag

SEQ ID NO: 5 - DNA, 16 bp, Artificial Sequence
  synthetic
ccgcgggctgctagag

SEQ ID NO: 6 - DNA, 20 bp, Artificial Sequence
  synthetic
ggcaactccggaaacttcct

SEQ ID NO: 7 - DNA, 15 bp, Artificial Sequence
  synthetic
ggtgcttggaggaaa

SEQ ID NO: 8 - DNA, 14 bp, Artificial Sequence
  synthetic
ggtgcttgcaggaa

SEQ ID NO: 9 - DNA, 18 bp, Artificial Sequence
  synthetic
tccatgcctgggcattcc

SEQ ID NO: 10 - DNA, 31 bp, Artificial Sequence
  synthetic
ccaaatactagactcaaattacagtcctcat

SEQ ID NO: 11 - DNA, 17 bp, Artificial Sequence
  synthetic
tcatttgccagtgattt

SEQ ID NO: 12 - DNA, 18 bp, Artificial Sequence
  synthetic
ctcatttgcctgtgattt

SEQ ID NO: 13 - DNA, 25 bp, Artificial Sequence
  synthetic
acagcttctctttggcataactgaa

SEQ ID NO: 14 - DNA, 27 bp, Artificial Sequence
  synthetic
tcagttttgcagtgtttgttttcaagt

SEQ ID NO: 15 - DNA, 18 bp, Artificial Sequence
  synthetic
ccaacgtcactactcttg

SEQ ID NO: 16 - DNA, 18 bp, Artificial Sequence
  synthetic
ccaatgtcactactcttg

SEQ ID NO: 17 - DNA, 25 bp, Artificial Sequence
  synthetic
gtggtgttgttgtagacaaaccttt

SEQ ID NO: 18 - DNA, 23 bp, Artificial Sequence
  synthetic
gcattctgtttcctacggttggt

SEQ ID NO: 19 - DNA, 17 bp, Artificial Sequence
  synthetic
gctttcgttttgtttgt

SEQ ID NO: 20 - DNA, 18 bp, Artificial Sequence
  synthetic
gctttcattttgtttgtg

SEQ ID NO: 21 - DNA, 31 bp, Artificial Sequence
  synthetic
tgtttgaagttagctttattaaggtgtcact

SEQ ID NO: 22 - DNA, 25 bp, Artificial Sequence
  synthetic
gggtggctatataactgcatacgtt

SEQ ID NO: 23 - DNA, 18 bp, Artificial Sequence
  synthetic
aaatttacctactttaca

SEQ ID NO: 24 - DNA, 20 bp, Artificial Sequence
  synthetic
aaatttaactactttacatg

SEQ ID NO: 25 - DNA, 29 bp, Artificial Sequence
  synthetic
aaggaatctttgaaagggaatctattggt

SEQ ID NO: 26 - DNA, 25 bp, Artificial Sequence
  synthetic
ccaagccactaactctctctatcct

SEQ ID NO: 27 - DNA, 17 bp, Artificial Sequence
  synthetic
caaattaggcacagaaa

SEQ ID NO: 28 - DNA, 19 bp, Artificial Sequence
  synthetic
agcaaattagacacagaaa

SEQ ID NO: 29 - DNA, 27 bp, Artificial Sequence
  synthetic
ggtggtcctgatatttctcaattctgt

SEQ ID NO: 30 - DNA, 24 bp, Artificial Sequence
  synthetic
caggaacaactttagagggcagtt

SEQ ID NO: 31 - DNA, 14 bp, Artificial Sequence
  synthetic
ctaccccagcaatc

SEQ ID NO: 32 - DNA, 18 bp, Artificial Sequence
  synthetic
aaaactactccagcaatc

SEQ ID NO: 33 - DNA, 21 bp, Artificial Sequence
  synthetic
acgcagtgtgtcttccttcac

SEQ ID NO: 34 - DNA, 29 bp, Artificial Sequence
  synthetic
tcgacctcatcaattacattttcatgaca

SEQ ID NO: 35 - DNA, 16 bp, Artificial Sequence
  synthetic
ctttgtcggacagagc

SEQ ID NO: 36 - DNA, 16 bp, Artificial Sequence
  synthetic
ctttgtcggccagagc

SEQ ID NO: 37 - DNA, 33 bp, Artificial Sequence
  synthetic
ggaaaaggaggaagaataaaaaatgcattctaa

SEQ ID NO: 38 - DNA, 23 bp, Artificial Sequence
  synthetic
aaatgcctgggtgtgtgtatgta

SEQ ID NO: 39 - DNA, 15 bp, Artificial Sequence
  synthetic
cctgagacgaagttg

SEQ ID NO: 40 - DNA, 15 bp, Artificial Sequence
  synthetic
cctgagaggaagttg

SEQ ID NO: 41 - DNA, 25 bp, Artificial Sequence
  synthetic
gctgacactggcatcttctaatctg

SEQ ID NO: 42 - DNA, 23 bp, Artificial Sequence
  synthetic
tgtccctgcttacagtagtctct

SEQ ID NO: 43 - DNA, 13 bp, Artificial Sequence
  synthetic
agtgaccgccccc

SEQ ID NO: 44 - DNA, 14 bp, Artificial Sequence
  synthetic
cagtgacccccccc

SEQ ID NO: 45 - DNA, 25 bp, Artificial Sequence
  synthetic
gagagctggtagtcttcattccatt

SEQ ID NO: 46 - DNA, 25 bp, Artificial Sequence
  synthetic
ctagaatcatggactgcttggaagt

SEQ ID NO: 47 - DNA, 17 bp, Artificial Sequence
  synthetic
ctactcatccgttggtg

SEQ ID NO: 48 - DNA, 18 bp, Artificial Sequence
  synthetic
cctactcatccattggtg

SEQ ID NO: 49 - DNA, 24 bp, Artificial Sequence
  synthetic
gctactctgagattccgtctgatg

SEQ ID NO: 50 - DNA, 20 bp, Artificial Sequence
  synthetic
ggtggtcttgggaggtctct

SEQ ID NO: 51 - DNA, 16 bp, Artificial Sequence
  synthetic
ctgaggatgcgttgca

SEQ ID NO: 52 - DNA, 16 bp, Artificial Sequence
  synthetic
ctgaggatgcattgca

SEQ ID NO: 53 - DNA, 16 bp, Artificial Sequence
  synthetic
ccagggcagcctttgc

SEQ ID NO: 54 - DNA, 23 bp, Artificial Sequence
  synthetic
ctaccaagcatctcaaggaagga

SEQ ID NO: 55 - DNA, 16 bp, Artificial Sequence
  synthetic
ctcctgaccttggctg

SEQ ID NO: 56 - DNA, 17 bp, Artificial Sequence
  synthetic
cctcctaaccttggctg

SEQ ID NO: 57 - DNA, 39 bp, Artificial Sequence
  synthetic
gcttatttgtattttctatatcatacatgcatcacttct

SEQ ID NO: 58 - DNA, 25 bp, Artificial Sequence
  synthetic
cgtgggtctgcttttcatttagttg

SEQ ID NO: 59 - DNA, 17 bp, Artificial Sequence
  synthetic
actttccttggtaatct

SEQ ID NO: 60 - DNA, 18 bp, Artificial Sequence
  synthetic
cactttccttagtaatct

SEQ ID NO: 61 - DNA, 31 bp, Artificial Sequence
  synthetic
aaatggaatatcacacatctacaaagaggtt

SEQ ID NO: 62 - DNA, 44 bp, Artificial Sequence
  synthetic
tttaggtaattttgtattttatagtttatggtaagctttgtttt

SEQ ID NO: 63 - DNA, 20 bp, Artificial Sequence
  synthetic
aataattctccaggttactg

SEQ ID NO: 64 - DNA, 21 bp, Artificial Sequence
  synthetic
aaataattctccagattactg

SEQ ID NO: 65 - DNA, 17 bp, Artificial Sequence
  synthetic
tcccgcagctccgaatg

SEQ ID NO: 66 - DNA, 16 bp, Artificial Sequence
  synthetic
cgcgcgttccctcttg

SEQ ID NO: 67 - DNA, 15 bp, Artificial Sequence
  synthetic
cctcgagcccgagcg

SEQ ID NO: 68 - DNA, 15 bp, Artificial Sequence
  synthetic
cctcgagccggagcg

SEQ ID NO: 69 - DNA, 25 bp, Artificial Sequence
  synthetic
ccgctacatagtcaggtgaagattg

SEQ ID NO: 70 - DNA, 23 bp, Artificial Sequence
  synthetic
tccatgcttcctacaaccacatc

SEQ ID NO: 71 - DNA, 18 bp, Artificial Sequence
  synthetic
cagaagcctctgcagaga

SEQ ID NO: 72 - DNA, 18 bp, Artificial Sequence
  synthetic
cagaagcctctacagaga

SEQ ID NO: 73 - DNA, 25 bp, Artificial Sequence
  synthetic
agagagaggcatatgactcaagtga

SEQ ID NO: 74 - DNA, 24 bp, Artificial Sequence
  synthetic
gtatcatcctgctcacagttccaa

SEQ ID NO: 75 - DNA, 14 bp, Artificial Sequence
  synthetic
cccaggagagagtc

SEQ ID NO: 76 - DNA, 15 bp, Artificial Sequence
  synthetic
tcccaggacagagtc

SEQ ID NO: 77 - DNA, 22 bp, Artificial Sequence
  synthetic
gccattctctctgcttggagta

SEQ ID NO: 78 - DNA, 24 bp, Artificial Sequence
  synthetic
cctgagcaagtgacaatctaacct

SEQ ID NO: 79 - DNA, 13 bp, Artificial Sequence
  synthetic
cccccctggaacc

SEQ ID NO: 80 - DNA, 15 bp, Artificial Sequence
  synthetic
ctccccactggaacc

SEQ ID NO: 81 - DNA, 25 bp, Artificial Sequence
  synthetic
gacctccttttcagtcttgttcaca

SEQ ID NO: 82 - DNA, 18 bp, Artificial Sequence
  synthetic
ctgggtgccaagctcaga

SEQ ID NO: 83 - DNA, 17 bp, Artificial Sequence
  synthetic
tttttgagggccaggtc

SEQ ID NO: 84 - DNA, 19 bp, Artificial Sequence
  synthetic
cctttttgaggtccaggtc

SEQ ID NO: 85 - DNA, 17 bp, Artificial Sequence
  synthetic
gccgcctcgtcgtagtc

SEQ ID NO: 86 - DNA, 13 bp, Artificial Sequence
  synthetic
tgggccgcctgca

SEQ ID NO: 87 - DNA, 13 bp, Artificial Sequence
  synthetic
cggaggagcaggc

SEQ ID NO: 88 - DNA, 13 bp, Artificial Sequence
  synthetic
cggaggaacaggc

SEQ ID NO: 89 - DNA, 18 bp, Artificial Sequence
  synthetic
ctcctggcctcccatagc

SEQ ID NO: 90 - DNA, 24 bp, Artificial Sequence
  synthetic
cctagctagagagttgcatgacat

SEQ ID NO: 91 - DNA, 13 bp, Artificial Sequence
  synthetic
cccaggcctctct

SEQ ID NO: 92 - DNA, 15 bp, Artificial Sequence
  synthetic
ctcccagacctctct

SEQ ID NO: 93 - DNA, 15 bp, Artificial Sequence
  synthetic
acgttgcacacgagg

(19) United States
(12) Patent Application Publication
(10) Pub. No.: US 2007/0254288 A1
(43) Pub. Date: Nov. 1, 2007

(54) Diagnostic methods for pain sensitivity and chronicity and for tetrahydrobiopterin-related disorders

(75) Inventors: Clifford J. Woolf, Newton, MA (US); Michael Costigan, Somerville, MA (US); Mitchell B. Max, Garrett Park, MD (US); Inna Belfer, Rockville, MD (US); Steven J. Atlas, Cambridge, MA (US); Albert Kingman, Bethesda, MD (US); Tianxia Wu, Potomac, MD (US); David Goldman, Potomac, MD (US)

Correspondence Address: CLARK & ELBING LLP, 101 FEDERAL STREET, BOSTON, MA, 02110, US

(73) Assignees: The General Hospital Corporation, Boston, MA (US); National Institutes of Health, Bethesda, MD (US)

(21) Appl. No.: 11/584,449

(22) Filed: Oct. 20, 2006

Related U.S. Application Data
(60) Provisional application No. 60/742,820, filed on Dec. 6, 2005.

(51) Int. Cl. C12Q 1/02; C12Q 1/68

(52) U.S. Cl. 435/6; 435/29

(57) ABSTRACT

Disclosed herein are methods for determining whether a subject possesses altered pain sensitivity an altered risk of developing acute or chronic pain, or diagnosing a tetrahydrobiopterin (BH4)-related disorder or a propensity thereto. These methods are based on the discovery of GCH1 and KCNS1 allelic variants that are associated with altered pain sensitivity and altered risk of developing acute or chronic pain, and the discovery that a GCH1 “pain protective haplotype” is associated with decreased upregulation of BH4 synthesis in treated leukocytes.

### CROSS-REFERENCE TO RELATED APPLICATIONS

This application claims benefit of U.S. Provisional Application No. 60/742,820, filed Dec. 6, 2005, which is hereby incorporated by reference.

### STATEMENT AS TO FEDERALLY FUNDED RESEARCH

The United States Government has a paid-up license in this invention and the right in limited circumstances to require the patent owner to license others on reasonable terms as provided for by the terms of NS039518, NS038253, Z01 DE00366, Z01 AA000301, DE16558, DE07509, and NS045685 awarded by the National Institutes of Health.

### BACKGROUND OF THE INVENTION

Clinical pain conditions, including inflammatory and neuropathic pain, and pain hypersensitivity syndromes without any clear tissue injury or lesion to the nervous system result from diverse neurobiological mechanisms operating in the peripheral and central nervous systems. Some mechanisms are unique to a particular disease etiology and others are common to multiple pain syndromes. Some mechanisms are transient and some irreversible (Scholz and Woolf, Nat Neurosci 5:1062-1067 (2002)). These include changes in the excitability and threshold of primary sensory neurons, alterations in synaptic processing in the spinal cord, loss of inhibitory interneurons, and modifications in brainstem facilitatory and inhibitory input to the spinal cord. These changes in neuronal activity result from novel gene transcription, posttranslational modifications, alterations in ion channel and receptor trafficking, activation of microglia, neuroimmune interactions, and neuronal apoptosis (Marchand et al., Nat Rev Neurosci 6:521-32 (2005); Woolf et al., Science 288:1765-1769 (2000); Tsuda et al., Trends Neurosci 28:101-107 (2005); Hunt and Mantyh, Nat Rev Neurosci 2:83-91 (2001); Scholz et al., J Neurosci 25:7317-7323 (2005)). Pain hypersensitivity, manifesting as spontaneous pain, pain in response to normally innocuous stimuli (allodynia), and an exaggerated response to noxious stimuli (hyperalgesia) are the dominant features of clinical pain and persist, in some individuals, long after the initial injury is resolved.

Several studies in inbred rodent strains and human twins suggest that the risk of developing chronic pain may be genetically determined (Mogil et al., Pain 80:67-82 (1999); Diatchenko et al., Hum Mol Genet 14:135-43 (2005); Norbury et al., 11th World Congress on Pain, Sydney, Australia Abstract (2005); Fillingim et al., J Pain 6:159-67 (2005); Zondervan et al., Behav Genet 35:177-88 (2005); MacGregor et al., Arthritis Rheum 51:160-7 (2004)). However, prior to the present invention, it was not well understood what perpetuates the maladaptive processes that sustain enhanced pain sensitivity in certain individuals. Neither were reliable predictors of pain response available.

### SUMMARY OF THE INVENTION

The invention provides methods and kits for predicting pain sensitivity, diagnosing the risk of developing acute or chronic pain based on the identification of pain protective allelic variants in the GCH1 and KCNS1 genes, or the risk of diagnosing an increased risk of developing a tetrahydrobiopterin (BH4)-related disorder in a mammalian subject, based on the identification of allelic variants in the GCH1 gene.

In one particular aspect, the invention features a method for predicting pain sensitivity, diagnosing the risk of developing acute or chronic pain, or diagnosing the risk of developing a BH4-related disorder (e.g., cardiovascular disease or any BH4-related disorder described herein) in a mammalian subject that includes determining the presence or absence of an allelic variant in a GTP cyclohydrolase (GCH1) nucleic acid in a biological sample from the subject, the allelic variant correlating with pain sensitivity, development of acute or chronic pain, or a BH4-related disorder. The GCH1 allelic variant may be present in a haplotype block located within human chromosome 14q22.1-14q22.2 (e.g., an allelic variant including a SNP selected from the group consisting of the SNPs listed in Table 1 or an allelic variant including an A at position C.-9610, a T at position C.343+8900, or both). In certain embodiments, the allelic variant may include an A at position C.-9610, C at position C.-4289, G at position C.343+26, T at position C.343+8900, T at position C.343+10374, G at position C.343+14008, C at position C.343+18373, A at position C.344-11861, C at position C.344-4721, A at position C.454-2181, C at position C.509+1551, G at position C.509+5836, A at position C.627-708, G at position C.*3932, and G at position C.*4279 of the GCH1 sequence (positions relative to the coding exons for the GCH1 gene, as shown in FIG. 11A)). The allelic variant may be present in a regulatory region (e.g., the promoter region, a 5′ regulatory region, a 3′ regulatory region, an enhancer element, or a suppressor element), within the coding region. (e.g., in an intron or in an exon) of the GCH1 gene, or any combination thereof. The cardiovascular disease may be atherosclerosis, ischemic reperfusion injury, cardiac hypertrophy, hypertension, vasculitis, myocardial infarction, or cardiomyopathy.

TABLE 1
SNPs identified in GCH1 (Data from the public NCBI SNP database)

| Contig position | dbSNP rs# | Heterozygosity | Validation | Function | dbSNP |
|---|---|---|---|---|---|
| 36308520 | rs6572984 | 0.014 | byCluster | untranslated | A/C |
| 36308570 | rs17128017 | 0.068 | byFreq | untranslated | A/G |
| 36309343 | rs10151500 | N.D. | | untranslated | C/T |
| 36309808 | rs10136966 | 0.01 | byFreqwithHapMapFreq | untranslated | C/T |
| 36310242 | rs841 | 0.414 | byClusterbyFreqbySubmitterHapMapFreq | untranslated | C/T |
| 36310244 | rs987 | N.D. | | untranslated | C/T |
| 36310875 | rs17253577 | 0.178 | byFreq | intron | C/T |
| 36310913 | rs11624963 | N.D. | withHapMapFreq | intron | A/G |
| 36311319 | rs752688 | N.D. | byCluster | intron | C/T |
| 36311729 | rs7493025 | N.D. | with2hit | intron | C/T |
| 36311808 | rs2004633 | N.D. | | intron | A/G |
| 36311808 | rs7493033 | N.D. | | intron | C/T |
| 36313081 | rs17253584 | 0.178 | byFreq | intron | C/T |
| 36313963 | rs10139369 | N.D. | with2hit | intron | A/T |
| 36314510 | rs10150825 | 0.078 | byFreqwithHapMapFreq | intron | C/G |
| 36314755 | rs11848732 | N.D. | with2hit | intron | C/T |
| 36315166 | rs17253591 | 0.119 | byFreq | intron | C/T |
| 36315425 | rs10143089 | 0.17 | byFreqwith2hitwithHapMapFreq | intron | C/T |
| 36315520 | rs13329045 | N.D. | | intron | C/T |
| 36315658 | rs10131232 | 0.5 | byFreqwith2hitwithHapMapFreq | intron | A/G |
| 36316020 | rs10133662 | N.D. | byClusterwith2hit | intron | A/G |
| 36316262 | rs10133941 | N.D. | byClusterwith2hit | intron | C/T |
| 36317163 | rs13329058 | N.D. | | intron | C/T |
| 36317667 | rs9672037 | N.D. | | intron | C/T |
| 36318096 | rs7161034 | N.D. | byClusterwith2hit | intron | A/C |
| 36318710 | rs7140523 | N.D. | | intron | C/T |
| 36319264 | rs11626298 | N.D. | with2hit | intron | A/G |
| 36319947 | rs17128021 | 0.178 | byFreq | intron | A/G |
| 36320020 | rs10129528 | 0.119 | byClusterbyFreq | intron | C/T |
| 36320313 | rs4411417 | N.D. | with2hit | intron | C/T |
| 36320535 | rs2878168 | 0.46 | byClusterbyFreqbySubmitteHapMapFreq | intron | A/G |
| 36320617 | rs11461307 | N.D. | | intron | —/T |
| 36322009 | rs7153186 | N.D. | | intron | A/G |
| 36322185 | rs7153566 | N.D. | | intron | A/G |
| 36322473 | rs7155099 | N.D. | | intron | G/T |
| 36322496 | rs11444305 | N.D. | | intron | —/A |
| 36322504 | rs11439363 | N.D. | | intron | —/A |
| 36322601 | rs7155309 | N.D. | | intron | C/T |
| 36323200 | rs1952437 | N.D. | with2hit | intron | A/G |
| 36324598 | rs8007201 | 0.5 | byClusterbyFreqwith2hitwithHapMapFreq | intron | A/G |
| 36324602 | rs11412107 | N.D. | | intron | —/T |
| 36325333 | rs12587434 | N.D. | with2hit | intron | G/T |
| 36325573 | rs17128028 | 0.068 | byFreq | intron | C/T |
| 36325612 | rs12589758 | N.D. | byClusterwith2hit | intron | A/T |
| 36325743 | rs2878169 | N.D. | | intron | G/T |
| 36326661 | rs28532361 | N.D. | | intron | C/T |
| 36326900 | rs12879111 | ND. | with2hit | intron | G/T |
| 36327073 | rs10129468 | N.D. | | intron | A/G |
| 36327209 | rs11620796 | N.D. | | intron | A/G |
| 36327287 | rs2149483 | N.D. | with2hit | intron | C/T |
| 36327806 | rs7147200 | 0.028 | byClusterbyFreq | intron | C/T |
| 36328179 | rs4462519 | N.D. | byClusterwith2hit | intron | A/G |
| 36328385 | rs9671371 | 0.476 | byClusterbyFreqwith2hitwithHapMapFreq | intron | C/T |
| 36328671 | rs9671850 | N.D. | with2hit | intron | A/T |
| 36328830 | rs9671455 | N.D. | | intron | C/G |
| 36329658 | rs28481447 | N.D. | | intron | C/T |
| 36329999 | rs12884925 | N.D. | | intron | A/T |
| 36330005 | rs8010282 | N.D. | | intron | A/G |
| 36330006 | rs8010689 | N.D. | | intron | A/G |
| 36330024 | rs8011751 | N.D. | | intron | C/T |
| 36331647 | rs7156475 | 0.069 | byClusterbyFreqwithHapMapFreq | intron | G/T |
| 36332549 | rs17128033 | 0.092 | byFreqwithHapMapFreq | intron | C/T |
| 36333108 | rs28643468 | N.D. | | intron | A/G |
| 36334812 | rs2183084 | N.D. | byClusterwith2hit | intron | C/G |
| 36334922 | rs10137881 | N.D. | | intron | A/G |
| 36335139 | rs2878170 | N.D. | | intron | A/G |
| 36335218 | rs12323905 | N.D. | | intron | C/T |
| 36335320 | rs10138301 | N.D. | | intron | A/G |
| 36335320 | rs12323579 | N.D. | with2hit | intron | A/G |
| 36335497 | rs10138429 | N.D. | | intron | A/G |
| 36335497 | rs12323582 | N.D. | | intron | A/G |
| 36336027 | rs7141433 | N.D. | byCluster | intron | C/T |
| 36336109 | rs7141483 | N.D. | byCluster | intron | C/T |
| 36336110 | rs7141319 | N.D. | byCluster | intron | A/G |
| 36336175 | rs2183083 | N.D. | | intron | A/G |
| 36336188 | rs2183082 | N.D. | byClusterwith2hit | intron | A/G |
| 36336501 | rs2183081 | 0.5 | byClusterbyFreqwith2hit | intron | C/T |
| 36336625 | rs7492600 | 0.439 | byClusterbyFreqwith2hitwithHapMapFreq | intron | G/T |
| 36336801 | rs8009470 | N.D. | with2hit | intron | A/C |
| 36336854 | rs10144581 | N.D. | | intron | A/G |
| 36336854 | rs12323758 | N.D. | | intron | A/G |
| 36337403 | rs10145097 | N.D. | | intron | A/G |
| 36337403 | rs13368101 | N.D. | | intron | A/G |
| 36337423 | rs10134163 | N.D. | | intron | C/T |
| 36337423 | rs13367062 | N.D. | with2hit | intron | C/T |
| 36337619 | rs4402455 | N.D. | | intron | G/T |
| 36337619 | rs7493427 | N.D. | with2hit | intron | G/T |
| 36337619 | rs10311834 | N.D. | | intron | G/T |
| 36337629 | rs9743836 | N.D. | | intron | A/G |
| 36337666 | rs4363780 | N.D. | | intron | A/G |
| 36337666 | rs7493265 | N.D. | with2hit | intron | A/G |
| 36337666 | rs10312723 | N.D. | | intron | A/G |
| 36337689 | rs4363781 | N.D. | | intron | A/G |
| 36337689 | rs7493266 | N.D. | byClusterwith2hit | intron | A/G |
| 36337689 | rs10312724 | N.D. | | intron | A/G |
| 36338006 | rs11627767 | N.D. | | intron | A/G |
| 36338071 | rs11850691 | N.D. | | intron | A/G |
| 36338090 | rs11627828 | N.D. | | intron | C/T |
| 36341827 | rs11626155 | N.D. | with2hit | intron | C/T |
| 36341863 | rs2878171 | N.D. | | intron | C/T |
| 36341911 | rs10220344 | N.D. | | intron | C/T |
| 36341911 | rs10782424 | N.D. | byCluster | intron | C/T |
| 36341993 | rs3965763 | N.D. | | intron | A/G |
| 36342727 | rs10146709 | N.D. | | intron | A/G |
| 36342817 | rs10146658 | N.D. | byCluster | intron | C/T |
| 36343449 | rs10147430 | 0.01 | byFreqwithHapMapFreq | intron | A/G |
| 36343629 | rs17128050 | 0.308 | byFreq | intron | C/T |
| 36343765 | rs12147422 | 0.443 | byFreqwith2hitwithHapMapFreq | intron | C/T |
| 36344651 | rs28477407 | N.D. | | intron | C/T |
| 36345448 | rs10143025 | N.D. | | intron | C/T |
| 36345820 | rs10133449 | N.D. | | intron | C/T |
| 36346023 | rs10133650 | N.D. | with2hit | intron | C/G |
| 36346352 | rs3945570 | N.D. | | intron | A/G |
| 36346421 | rs28757745 | N.D. | | intron | A/C |
| 36346523 | rs28542181 | N.D. | | intron | C/T |
| 36347577 | rs7155501 | N.D. | byClusterwith2hit | intron | A/G |
| 36347666 | rs3825610 | N.D. | | intron | A/T |
| 36347868 | rs3783637 | 0.36 | byClusterbyFreqwithHapMapFreq | intron | C/T |
| 36348123 | rs3783638 | 0.401 | byClusterbyFreqwith2hit | intron | A/G |
| 36348416 | rs3783639 | 0.301 | byFreq | intron | C/T |
| 36348587 | rs3825611 | N.D. | byCluster | intron | C/G |
| 36348619 | rs11158026 | N.D. | with2hit | intron | C/T |
| 36348853 | rs11158027 | N.D. | byClusterwith2hit | intron | C/T |
| 36349008 | rs10873086 | N.D. | byClusterwith2hit | intron | C/T |
| 36349299 | rs11626210 | N.D. | | intron | C/T |
| 36350416 | rs8004445 | N.D. | with2hit | intron | G/T |
| 36350446 | rs8004018 | 0.44 | byFreqwith2hitwithHapMapFreq | intron | A/G |
| 36350935 | rs8010461 | N.D. | | intron | G/T |
| 36351248 | rs9805909 | N.D. | | intron | A/C |
| 36351267 | rs8009759 | N.D. | byClusterwith2hit | intron | A/C |
| 36351864 | rs10444720 | N.D. | | intron | A/G |
| 36352271 | rs4901549 | N.D. | with2hit | intron | C/T |
| 36352271 | rs3783640 | N.D. | | intron | C/T |
| 36352613 | rs10136545 | N.D. | with2hit | intron | C/T |
| 36352937 | rs10139282 | N.D. | byClusterwith2hit | intron | A/G |
| 36353118 | rs8020798 | N.D. | | intron | C/T |
| 36353467 | rs10498471 | 0.287 | byFreq | intron | A/G |
| 36353538 | rs28417208 | N.D. | | intron | A/T |
| 36354490 | rs11845055 | N.D. | | intron | G/T |
| 36354619 | rs10498472 | 0.072 | byClusterbyFreqwithHapMapFreq | intron | G/T |
| 36354781 | rs998259 | 0.184 | byClusterbyFreqbySubmitterwithHapMapFreq | intron | C/T |
| 36354821 | rs8011712 | N.D. | | intron | C/G |
| 36354999 | rs11312854 | N.D. | | intron | —/G |
| 36355164 | rs11410453 | N.D. | | intron | —/T |
| 36355411 | rs10782425 | N.D. | byCluster | intron | A/G |
| 36356144 | rs10149080 | N.D. | | intron | C/T |
| 36356275 | rs17128052 | 0.308 | byFreq | intron | C/G |
| 36357521 | rs8003903 | N.D. | | intron | C/T |
| 36357570 | rs10645822 | N.D. | | intron | —/TTTG |
| 36357997 | rs10132356 | N.D. | | intron | C/T |
| 36357997 | rs13366912 | N.D. | | intron | C/T |
| 36358389 | rs12885400 | N.D. | | intron | C/T |
| 36358415 | rs7147286 | 0.497 | byFreqwith2hitwithHapMapFreq | intron | A/G |
| 36358505 | rs7147040 | N.D. | | intron | C/T |
| 36358627 | rs7147201 | N.D. | with2hit | intron | A/G |
| 36359572 | rs17832263 | 0.106 | byFreq | intron | A/G |
| 36359806 | rs10133661 | 0.07 | byClusterbyFreq | intron | C/T |
| 36359889 | rs3783641 | 0.393 | byClusterbyFreqwithHapMapFreq | intron | A/T |
| 36359953 | rs3783642 | 0.5 | byClusterbyFreqwith2hitwithHapMapFreq | intron | C/T |
| 36360420 | rs12432756 | N.D. | | intron | G/T |
| 36360595 | rs10134429 | N.D. | | intron | G/T |
| 36361212 | rs10598935 | N.D. | | intron | —/AA |
| 36361215 | rs10545051 | N.D. | | intron | —/AA |
| 36361421 | rs17128057 | 0.041 | byFreq | intron | C/T |
| 36361522 | rs8016730 | N.D. | | intron | A/C |
| 36361586 | rs8017210 | 0.385 | byClusterbyFreqwith2hit | intron | A/G |
| 36362770 | rs11844799 | N.D. | | intron | A/G |
| 36362919 | rs12883072 | N.D. | | intron | G/T |
| 36363071 | rs10131633 | N.D. | with2hit | intron | A/G |
| 36363151 | rs10131563 | N.D. | | intron | C/T |
| 36364781 | rs10149945 | 0.074 | byClusterbyFreqwith2hitwithHapMapFreq | intron | G/T |
| 36365022 | rs8019791 | 0.096 | byFreqwithHapMapFreq | intron | C/T |
| 36365081 | rs8019824 | N.D. | byClusterwith2hit | intron | A/T |
| 36365131 | rs8018688 | N.D. | byClusterwith2hit | intron | A/G |
| 36365639 | rs10138594 | N.D. | | intron | A/C |
| 36366032 | rs10141456 | N.D. | byClusterwith2hit | intron | A/G |
| 36366637 | rs9972204 | N.D. | | intron | A/G |
| 36368377 | rs2149482 | N.D. | with2hit | intron | A/G |
| 36368645 | rs28413055 | N.D. | | intron | A/G |
| 36368736 | rs2183080 | 0.074 | byFreqwithHapMapFreq | intron | C/G |
| 36369171 | rs28458175 | N.D. | | untranslated | A/G |
| 36369252 | rs1753589 | 0.036 | | untranslated | C/T |

In another aspect, the invention features a method for predicting pain sensitivity or diagnosing the risk of developing acute or chronic pain in a mammalian subject that includes determining the presence or absence of an allelic variant in a potassium voltage-gated channel, delayed-rectifier, subfamily S, member 1 (KCNS1) nucleic acid in a biological sample from the subject, the allelic variant correlating with pain sensitivity or development of acute or chronic pain. The KCNS1 allelic variant may be present in a haplotype block located within human chromosome 20q12, may cause altered (e.g., increases or decreased) activity, expression, heteromultimerization, or trafficking of the KCNS1 protein. The allelic variant may be present in a regulatory region (e.g., the promotor region a 5′ regulatory region, a 3′ regulatory region, an enhancer element, or a suppressor element), within the coding region (e.g., in an intron or in an exon) of the KCNS1 gene, or any combination thereof. The allelic variant may include a SNP selected from the group consisting of the SNPs listed in Table 2 or may include an A at position 43,157,041 (e.g., include a G at position 43,155,431, A at position 43,157,041, and C at position 43,160,569) of the KCNS1 sequence (positions from SNP browser software and the Panther Classification System public database, November 2005).

TABLE 2
SNPs identified in KCNS1 (Data from the public NCBI SNP database)

| Contig | | | | | | | | Amino |
|---|---|---|---|---|---|---|---|---|
| position | dbSNP rs# | Heterozygosity | Validation | Function | dbSNP | Protein | Codon | acid |
| 8774296 | rs6124683 | N.D. | | Untranslated | C/T | | | |
| 8774334 | rs4499491 | N.D. | with2hit | Untranslated | A/C | | | |
| 8774377 | rs8118000 | N.D. | | Untranslated | A/G | | | |
| 8774408 | rs6124684 | 0.239 | byFreqwithHapMapFreq | Untranslated | C/T | | | |
| 8774434 | rs6124685 | N.D. | | Untranslated | C/T | | | |
| 8774659 | rs12480253 | N.D. | | Untranslated | C/G | | | |
| 8774680 | rs6124686 | N.D. | | Untranslated | C/T | | | |
| 8774932 | rs6124687 | 0.151 | byFreq | Untranslated | G/T | | | |
| 8775044 | rs6031988 | N.D. | | Untranslated | A/C | | | |
| 8775190 | rs6065785 | N.D. | | Untranslated | C/T | | | |
| 8775491 | rs1054136 | N.D. | | Untranslated | C/T | | | |
| 8775491 | rs17341034 | N.D. | | Untranslated | C/T | | | |
| 8776002 | rs6031989 | N.D. | | Untranslated | C/T | | | |
| 8776484 | rs7264544 | 0.014 | byFreqwith2hitwithHapMapFreq | nonsynonymous | G | Arg [R] | 2 | 508 |
| | | 0.014 | byFreqwith2hitwithHapMapFreq | contig reference | A | Gln [Q] | 2 | 508 |
| 8776542 | rs734784 | 0.464 | byFreqbySubmitterHapMapFreq | nonsynonymous | G | Val [V] | 1 | 489 |
| | | 0.464 | byFreqbySubmitterHapMapFreq | contig reference | A | Ile [I] | 1 | 489 |
| 8777122 | rs6104003 | N.D. | | Intron | A/G | | | |
| 8777133 | rs6104004 | N.D. | | Intron | A/G | | | |
| 8777159 | rs11699337 | N.D. | | Intron | A/G | | | |
| 8777794 | rs6017486 | 0.341 | byFreqwith2hitwithHapMapFreq | Intron | A/G | | | |
| 8778642 | rs962550 | N.D. | with2hit | Intron | A/G | | | |
| 8779347 | rs7261171 | N.D. | | Synonymous | T | Gly [G] | 3 | 327 |
| | | N.D. | | contig reference | C | Gly [G] | 3 | 327 |
| 8780057 | rs6104005 | N.D. | | Synonymous | T | Leu [L] | 1 | 91 |
| | | N.D. | | contig reference | C | Leu [L] | 1 | 91 |
| 8780070 | rs13043825 | N.D. | | synonymous | A | Glu [E] | 3 | 86 |
| | | N.D. | | contig reference | G | Glu [E] | 3 | 86 |
| 8780525 | rs7360359 | N.D. | | intron | G/T | | | |
| 8780563 | rs8192648 | N.D. | | intron | A/G | | | |
| 8780597 | rs6073642 | N.D. | | intron | A/G | | | |
| 8780860 | rs6130749 | N.D. | | untranslated | A/G | | | |
| 8780985 | rs6073643 | N.D. | byClusterwith2hit | untranslated | C/T | | | |
| 8781005 | rs6104006 | N.D. | | untranslated | C/T | | | |
| 8781347 | rs6031990 | N.D. | | untranslated | A/G | | | |
| 8782397 | rs8122867 | N.D. | | untranslated | G/T | | | |
| 8782579 | rs8123330 | N.D. | | untranslated | C/G | | | |
| 8782586 | rs3213543 | N.D. | | untranslated | C/T | | | |

In either of the above aspects, the method may include determining whether the nucleic acid sample includes one copy or multiple copies of the allelic variant. The acute pain may be one or more of mechanical pain, heat pain, cold pain, ischemic pain, or chemical-induced pain. The pain may also be peripheral or central neuropathic pain, inflammatory pain, headache pain (e.g., migraine-related pain), irritable bowel syndrome-related pain, fibromyalgia-related pain, arthritic pain, skeletal pain, joint pain, gastrointestinal pain, muscle pain, angina pain, facial pain, pelvic pain, claudication, postoperative pain, post traumatic pain, tension-type headache, obstetric or gynecological pain, or chemotherapy-induced pain. The mammal may be a human.

The presence or absence of the allelic variant may be determined by nucleic acid sequencing or by PCR analysis. In addition, the method may be used to determine the dosing or choice of an analgesic or an anesthetic administered to the subject; whether to include the subject in a clinical trial involving an analgesic; whether to carry out a surgical procedure (e.g., a surgical procedure involving nerve damage or treatment of nerve damage) on the subject; or whether to administer a neurotoxic treatment to the subject. Further, the method may be used to determine the likelihood of pain development in the subject as part of an insurance risk analysis or as criterion for a job assignment. The method may also be used in conjunction with a clinical trial, for example, as a basis for establishing a statistical significant difference between the control group and the experimental group in a clinical trial involving pain or another disorder involving GCH1 such as those described herein.

In either of the above aspects, the allelic variants in Tables 1 and 2 represent exemplary SNPs that may be utilized to predict a subject's pain profile; alternative selection of one or more SNPs may also be used to identify a pain protective phenotype, and these one or more SNPs may be extended beyond the genomic regions described in detail herein. In addition to SNPs, other types of genetic variation (e.g., variable number tandem repeats (VNTRs), or short tandem repeats (STRs)) may be used in the methods of the invention. Such sequences may be derived from public or commercial databases. Novel SNPs may be identified by resequencing of gene regions; such novel SNPs also may be used in the methods of the invention.

The methods of the invention may be performed using any genotyping assay, e.g., those described herein. The methods may further be combined with genotyping for polymorphisms in additional genes known or identified to affect the risk of developing pain (e.g., COMT).

The methods of the invention may employ any genotyping method for identification of human genotypes, haplotypes, or diplotypes. A wide range of methods is known in the art, including chemical assays (e.g., allele specific hybridization, polymerase extension, oligonucleotide ligation, enzymatic cleavage, flap endonuclease discrimination) and detection methods (e.g., fluorescence, colorimetry, chemiluminiscence, and mass spectrometry). Specific methods are described herein. Desirably, a genotyping method is robust, highly sensitive and specific, rapid, amenable to multiplexing and high-throughput analysis, and of reasonable cost.

In a third aspect, the invention features a method for predicting pain sensitivity, diagnosing the risk of developing acute or chronic pain, or diagnosing the risk of developing a BH4-associated disorder in a mammalian subject. The method includes the steps of (a) contacting a biological sample including a cell (e.g., a smooth muscle cell, an endothelial cell, a vascular cell, a lymphocyte, or a leukocyte) from the subject with a sufficient amount of a composition that (i) increases the level of cyclic AMP in the cell (e.g., a phosphodiesterase inhibitor, an adenyl cyclase activator such as forskolin, or a cAMP, analog such as those described herein), (ii) includes lipopolysaccharide (LPS), or (iii) includes an inflammatory cytokine (e.g., tumor necrosis factor α, interleukin-1β, and interferon-γ); and (b) measuring the expression or activity of GTP cyclohydrolase (GCH1) in the sample, wherein the level of said expression or activity, when compared to a baseline value, is indicative of whether said patient has altered (e.g., increased or decreased) pain sensitivity or is diagnostic of the risk of developing acute or chronic pain or developing a BH4-associated disorder in said subject. A decrease in GCH1 expression or activity relative to a baseline value may be indicative of decreased pain sensitivity or decreased risk of developing acute or chronic pain. GCH1 expression may be measured by determining GCH1 mRNA or GCH1 protein level in the cell. GCH1 activity may be measured by determining neopterin, biopterin, or BH4 levels in the cell.

In a fourth aspect, the invention features a kit for predicting pain sensitivity, diagnosing the risk of developing acute or chronic pain, or diagnosing a propensity to develop a BH4-related disorder in a mammalian subject that includes a set of primers for amplification of a sequence including an allelic variant in a GCH1 gene, and instructions for use. The GCH1 allelic variant may be present in a haplotype block located within human chromosome 14q22.1-14q22.2 (e.g., the GCH1 allelic variant may include a SNP selected from the group consisting of the SNPs listed in Table 1 or the GCH1 allelic variant may include an A at position C.-9610, a T at position C.343+8900, or both). In certain embodiments, the allelic variant may include an A at position C.-9610, C at position C.-4289, G at position C.343+26, T at position C.343+8900, T at position C.343+10374, G at position C.343+14008, C at position C.343+18373, A at position C.344-11861, C at position C.344-4721, A at position C.454-2181, C at position C.509+1551, G at position C.509+5836, A at position C.627-708, G at position C.*3932, and G at position C.*4279 of the GCH1 sequence (positions relative to the exons in the GCH1 gene, as shown in FIG. 11A)). The allelic variant may be present in the promoter region, within a coding region (e.g., in an intron or in an exon), in a 5′ or 3′ regulatory region of the GCH1 gene, or any combination thereof.

In a fifth aspect, the invention features a kit for predicting pain sensitivity or diagnosing the risk of developing acute or chronic pain in a mammalian subject that includes a set of primers for amplification of a sequence including an allelic variant in a KCNS1 gene and instructions for use. The KCNS1 allelic variant may be present in a haplotype block located within human chromosome 20q12. The KCNS1 allelic variant may cause altered (e.g., decreased) activity, expression, heteromultimerization, or trafficking of the KCNS1 protein; the allelic variant may include a SNP selected from the group consisting of the SNPs in Table 2 or may include an A at position 43,157,041 (e.g., a G at position 43,155,431, A at position 43,157,041, and C at position 43,160,569) of the KCNS1 sequence (positions from the SNP browser software and the Panther Classification System public database, November 2005).

In a sixth aspect, the invention features a kit for predicting pain sensitivity, diagnosing the risk of developing acute or chronic pain, or diagnosing the risk of developing an BH4-related disorder in a mammalian subject. The kit includes (i) an agent for increasing cyclic AMP levels in a cell, (ii) LPS, or (iii) an inflammatory cytokine (e.g., those described herein); an antibody specific for GTP cyclohydrolase (GCH1); a first primer for hybridization to a GTP cyclohydrolase (GCH1) mRNA sequence; and instructions for use. The kit may further include a second primer, where the first and second primers are capable of being used to amplify at least a portion of the GCH1 mRNA sequence.

In a seventh aspect, the invention features a kit for predicting pain sensitivity, diagnosing the risk of developing acute or chronic pain, or diagnosing the risk of developing an BH4-related disorder in a mammalian subject. The kit includes (i) an agent for increasing cyclic AMP levels in a cell, (ii) LPS, or (iii) an inflammatory cytokine (e.g., those described herein); an antibody specific for GTP cyclohydrolase (GCH1); and instructions for use.

In either the sixth or seventh aspect of the invention, the agent may be an adenyl cyclase activator (e.g., forskolin), a phosphodiesterase inhibitor, or any agent described herein.

As used herein, by “pain sensitivity” is meant the threshold, duration or intensity of a pain sensation including the sensation of pain in response to normally non-painful stimuli and an exaggerated or prolonged response to a painful stimulus.

By “biological sample” is meant a tissue biopsy, cell, bodily fluid (e.g., blood, serum, plasma, semen, urine, saliva, amniotic fluid, or cerebrospinal fluid) or other specimen obtained from a patient or a test subject.

By “increase” is meant a positive change of at least 3% as compared to a control value or baseline level. An increase may be at least 5%, 10%, 20%, 30%, 50%, 75%, 100%, 150%, 200%, 500%, 1,000% as compared to a control value.

By “decrease” is meant a negative change of at least 3% as compared to a control value or baseline level. A decrease may be at least 5%, 10%, 20%, 30%, 50%, 60%, 70%, 80%, 90%, 95%, or 99%, or even 100% as compared to a control value.

By “allelic variant” or “polymorphism” is meant a segment of the genome that is present in some individuals of a species and absent in other individuals of that species. Allelic variants can be found in the exons, introns, or the coding region of the gene or in the sequences that control expression of the gene.

By “baseline value,” is meant value to which an experimental value may be compared. Depending on the assay, the baseline value can be a positive control (e.g., from an individual known to possess a pain protective haplotype). In certain cases, it may be desirable to calculate the baseline value from an average over a population of individuals (e.g., individuals selected at random or individuals selected who possess or lack a particular genetic background, such as zero, one, or two copies of the GCH1 pain protective haplotype). One of skill in the art will know which baseline value is appropriate for the desired comparison and how to calculate such baseline values. Exemplary baseline values and means for determining such values for use in the methods of the invention are described herein.

By “BH4-related disorder” is meant any disease or condition caused by an increase or decrease in BH4 expression, concentration, or activity. Such disorders include any disease related to endothelial cell function such as cardiovascular disease including atherosclerosis, ischemic reperfusion injury, cardiac hypertrophy, vasculitis, hypertension (e.g., systemic or pulmonary), myocardial infarction, and cardiomyopathy. Increased risk of developing a BH4-related disorder is associated with individuals having a sedentary lifestyle, hypertension, hypercholesterolemia, diabetes mellitus, or chronic smoking. BH4 is involved in nitric oxide, 5-HT, dopamine, and nor-epinephrine, production, and any diseases or disorders involving these neurotransmitters, particularly in the cardiovascular and nervous systems, are encompassed by the term BH4-related disorder. For example, a GCH1 haplotype may be a marker for the risk of developing CVS disease (e.g., atherosclerosis, hypertension, myocardial infarction, or cardiomyopathy) as well as nervous system diseases other than pain. BH4-related disorders thus include diabetes, depression, neurodegenerative disorders (e.g., Parkinson's disease, Alzheimer's disease, amyotrophic lateral sclerosis, Huntington's disease, multiple sclerosis), schizophrenia, carcinoid heart disease, and autonomic disturbance, or dystonia.

The use of GCH1 and KCNS1 polymorphisms as predictors of the intensity and chronicity or persistence of pain is a powerful tool that can be used to assist treatment decisions, including estimation of the risk-benefit ratio of a medical procedure, for example, surgery involving or treating nerve damage, neurotoxic treatments for cancer or HIV infection. Further, such diagnostic methods may be used to determine the need for aggressive analgesic treatment for patients with increased risk of developing acute or chronic pain or for avoiding damage to nerves in surgery. The methods may be used for determining whether a patient is at an increased risk of developing disorders related to endothelial cell function, including cardiovascular diseases. The methods may also be utilized in clinical trial design, for example, to determine whether to include a subject in a trial involving or testing an analgesic or analgesic procedure. Further, the method may be used, for example, by one in the insurance industry as part of a risk analysis profile for a subject's response to pain or therapy or for a determination of the subject's likelihood (e.g., by a current or potential employer or by an insurance company) of developing an inappropriate pain response.

Other features and advantages of the invention will be apparent from the following Detailed Description, the drawings, and the claims.

### BRIEF DESCRIPTION OF THE DRAWINGS

FIG. 1 shows regulation of mRNA expression of BH4-dependent enzymes: phenylalanine hydroxylase (PheOH), tyrosine hydroxylase (TyrOH), neuronal tryptophan hydroxylase (nTrpOH), and endothelial, inducible, and neuronal nitric oxide synthases (eNOS, iNOS, and NNOS) in dorsal root ganglia (DRGs) in the spared nerve injury (SNI) model (3 days, n=3, error SEM; *p<0.05 versus vehicle).

FIGS. 2A-2H show regulation of tetrahydrobiopterin synthesizing enzymes in DRGs after nerve injury. FIG. 2A shows upregulation of BH4 synthetic pathway enzymes in L4/5 DRGs in the spared nerve injury (SNI) model of peripheral neuropathic pain, as detected by Affymetrix RGU34A microarrays (n=3, error SEM). Univariate ANOVA was consistent with differential expression of GTP cyclohydrolase (GTPCH) and sepiapterin reductase (SR) (p<0.001). Pyrovoyl-tetrahydropterin synthase (PTPS) was unchanged (data not shown). FIG. 2B shows the BH4 synthetic pathway. FIG. 2C shows validation of the increase in GTPCH, SR, and dihydropteridine reductase (DHPR) (also called quinoid dihydropteridine reductase (QDPR)) mRNA in L5 DRG neurons by in situ hybridization 7 days after SNI (Scale bar 100 μm). FIG. 2D shows GTPCH protein expression in L4/5 DRGs after SNI (n=3, error SEM). FIGS. 2E and 2F show neopterin and biopterin levels, respectively, in ipsi- and contralateral L4/5 DRGs 7 days after SNI. The GTPCH inhibitor 2,4-diamino-6-hydroxypyrimidine (DAHP) (single dose of 180 mg/kg i.p.) administered 3 hours before tissue dissection reduced neopterin and biopterin (n=6, error SEM). FIG. 2G shows in situ and immuno images three days after SNI; GTPCH mRNA positive neurons also label for the transcription factor ATF-3, a marker for neurons with injured axons For all panels * p<0.05. FIG. 2H shows upregulation of BH4 producing enzymes in L4/5 DRG neurons in the spared nerve injury (SNI) model of peripheral neuropathic pain as detected by quantitative RT-PCR (n=4, error SEM).

FIGS. 3A-3E show microarray analysis. FIGS. 3A and 3B show Affymetrix microarry analysis (n=3, error SEM) of GTP cyclohydrolase (GTPCH), sepiapterin reductase (SR) and dihydropteridine reductase (DHPR/QDPR) mRNA expression in L4/5 DRGs in the chronic constriction injury model (CCI; p<0.05 for GTPCH and SR) and analgesic effects of the GTP cyclohydrolase inhibitor, DAHP after CCI. FIGS. 3C and 3D show microarray analysis (n=3, error SEM; p<0.001 for GTPCH and SR, p=0.01 for DHPR) and analgesic effects of DAHP in the spinal nerve ligation model (SNL) of neuropathic pain. DAHP (180 mg/kg i.p.) was injected at the indicated days; n=9-10, p <0.05 for CCl and SNL. FIG. 3E shows microarray analysis of GCH1, SPR, and QDPR mRNA in ipsilateral lumbar DRGs in the complete Freund's adjuvant (CFA) (FIG. 3E) induced paw inflammation model. Control animals were treated with vehicle. Effect versus time AUCs were used for statistical comparisons of behavioral effects. For all panels, error is SEM.

FIGS. 4A-4D show upregulation of BH4 synthesis pathway enzymes in the L4/5 DRGs following sciatic nerve section. FIG. 4A is a table showing Affymetrix microarray analysis (n=3, error SEM). FIG. 4B shows Northern blot analysis of GTP cyclohydrolase (GTPCH), sepiapterin rductase (SR), and dihydropteridine reductase (DHPR/QDPR) mRNA over time (n=3, error SD). FIG. 4C shows GTP cyclohydrolase protein expression (n 3, error SD). FIG. 4D shows persistent GTPCH protein upregulation 40 days after sciatic nerve section (n=3, error SD).

FIG. 5 shows that some DRG neurons expressing GTP cyclohydrolase (GTPCH) mRNA colocalized with neurofilament 200 (NF200) three days after spared nerve injury (SNI; 40-50%). NF200 is a marker for large DRG neurons with myelinated axons. GTPCH mRNA expressing neurons were not labeled with Griffonia simplicifolia isolectin B4 (IB4), which is a marker for a subset of the small DRG neurons with unmyelinated axons. Arrows indicate neurons positive for the GTPCH transcript and NF200.

FIGS. 6A-6G show efficacy of the GTP cyclohydrolase inhibitor 2,4-diamino-6-hydroxy-pyrimidine (DAHP) in inflammatory and formalin induced pain. FIGS. 6A and 6B show that injection of DAHP (180 mg/kg i.p., arrow) significantly reduced thermal hyperalgesia induced by complete Freund's adjuvant (CFA) injection into the hindpaw both when it was injected before CFA (FIG. 6A) and 24 hours after CFA (FIG. 6B; n=7 or 9, p<0.05). FIGS. 6C and 6D show neopterin and biopterin levels, respectively, in ipsilateral L4/5 DRGs 24 h after CFA. DAHP (single dose of 180 mg/kg i.p.) administered 3 hours before tissue dissection reduced neopterin and biopterin (n=7, error SEM). FIG. 6E shows DAHP (180 mg/kg i.p.) injected before formalin (arrow) significantly reduced formalin-induced flinching behavior in both phases of the formalin assay (n=7, p<0.05). FIGS. 6F and 6G show the reduced number of cFOS immunoreactive neurons in the ipsilateral dorsal horn. For all figures, error SEM. The areas under the effect versus time curves were used for statistical comparisons of drug effects after CFA, the sum of flinches was used for the formalin test.

FIGS. 7A-7F show efficacy and kinetics of DAHP in the spared nerve injury (SNI) model of neuropathic pain. FIG. 7A shows that injection of DAHP four days after SNI (180 mg/kg i.p., arrow) significantly reduced mechanical (von Frey) and cold allodynia (n=12, p<0.05). FIG. 7B shows dose dependent efficacy of DAHP on mechanical and cold allodynia with repeated daily injections (arrows) in the SNI model, measured two-three hours after injection, (n=9-10, p<0.05). The relationship between dose and effect was linear (R=0.709 and R=0.754 for mechanical and cold allodynia, p<0.001). FIG. 7C shows that DAHP (180 mg/kg/d i.p.) treatment starting 17 days after nerve injury produced a significant reduction of mechanical and cold pain hypersensitivity (n=7, p<0.05). FIG. 7D shows that DAHP plasma and CSF concentration time courses after i.p. injection of 180 mg/kg. FIGS. 7E and 7F show DAHP (180 mg/kg i.p. arrow) treatment failed to modify mechanical and thermal threshold in naïve animals (n=6, p=1). For all figures, error SEM. The areas under the effect versus time curves were used for statistical comparisons of drug effects in behavioral experiments.

FIGS. 8A-8D show the effects of DAHP injection. FIGS. 8A and 8B show that continuous intrathecal infusion of DAHP reduced mechanical and cold allodynia in the SNI model of neuropathic pain. DAHP (250 μg/kg/h) was delivered to the lumbar spinal cord via a chronically implanted spinal catheter connected to an osmotic Alzet pump. Infusion started right after SNI surgery and continued 14 days, flow rate 5 μl/h (n=8, p<0.05). FIG. 8C shows that a single intrathecal injection of 1 mg/kg DAHP (arrow) reduced thermal hyperalgesia in the CFA induced paw inflammation model (n=9, p<0.05). Effect versus time AUCs were used for statistical comparisons. FIG. 8D shows the effects of DAHP in the Forced Swim Test. Rats (n=7 per condition) received 3 separate injections of DAHP (180 mg/kg, i.p.), at 1 hr, 19 hrs, and 23 hrs after the first exposure to forced swimming. This commonly used treatment regimen identifies in rats agents with antidepressant or pro-depressant effects in humans (Mague et al., J Pharmacol Exp Ther 305:323-330 (2003)). Retest sessions (forced swim for 300 sec) occurred 24 hr after the first swim exposure and were videotaped from the side of the water cylinders and scored by raters unaware of the treatment condition. Rats were rated at 5 sec intervals throughout the duration of the retest session; at each 5 sec interval the predominant behavior was assigned to one of four categories: immobility, swimming, climbing, or diving. The sum of these scores are shown for each modality. For all panels, error SEM.

FIGS. 9A-9I show the effects of N-acetyl serotonin (NAS) and BH4 in nerve injury and inflammatory models. FIGS. 9A and 9B show that the sepiapterin reductase inhibitor NAS (100 μg/kg/h i.t. infusion 14 days) significantly reduced mechanical and cold allodynia in the SNI model (n=9, p<0.05). FIG. 9C shows that NAS (50 mg/kg i.p.; arrow) injected 24 h after CFA significantly reduced thermal hyperalgesia (n=9, p<0.05), and FIG. 9D shows that NAS reduced biopterin levels in the DRGs seven days after SNI (n=8, *p<0.05). FIG. 9E-9H show that intrathecal injection of 6R-BH4 (10 μg, 10 μl, arrow) using a lumbar spinal catheter significantly increased heat pain sensitivity in naïve animals (n=6, p<0.05), further increased mechanical (FIG. 9F) and cold allodynia (FIG. 9G) six days after SNI and further increased (FIG. 9H) heat pain sensitivity when injected i.t. 5 days after CFA (n=6, for mechanical allodynia and heat hyperalgesia p<0.05). The increase of cold allodynia was not significant (n=6, p=0.15). FIG. 9I shows that neopterin, the stable metabolite produced during BH4 synthesis, had no effect on mechanical and thermal pain sensitivity in naïve rats after i.t. injection (10 μg, 10 μl, arrow). For all figures, error SEM. The areas under the effect versus time curves were used for statistical comparisons.

FIGS. 10A-10F show regulation of BH4-dependent enzymes in the DRG after nerve injury. FIG. 10A shows upregulation of neuronal tryptophan hydroxylase (TPH2) and neuronal nitric oxide synthase (NOS1) in L4/5 DRGs in the spared nerve injury (SNI) model of peripheral neuropathic pain as detected by quantitative RT-PCR (n=4, error SEM). FIG. 10B shows downregulation of tyrosine hydroxylase (TH) in L4/5 DRGs after SNI and no change of inducible and endothelial NOS(NOS2, NOS3) and phenylalanine hydroxylase (PAH) as detected by quantitative RT-PCR (n=3, error SEM). FIG. 10C shows increase of nitric oxide production in L4/5 DRGs 7 d after SNI and normalization of NO levels by once daily treatment with DAHP (n=6, error SEM). FIG. 10D shows the effects of the NOS inhibitor L-NAME (25 mg/kg i.p.) on SNI induced mechanical and cold allodynia seven days after nerve injury. L-NAME or vehicle was injected at time “zero” (n=7, error SEM). T-tests using AUCs showed significant effects for von Frey and acetone responses. FIG. 10E shows dose-dependent increase of intracellular calcium in cultured adult rat DRG neurons following application of 6R-BH4. [Ca²⁺]_{I} was measured fluorometrically in neurons loaded with fura-2 as absorbance ratio at 340 to 380 nm (AF 340/380). Blue-green-red pseudocolor radiometry images (upper panels) and representative ΔF340/380 trace from the neuron marked (*) demonstrate increases of ΔF after application of BH4. FIG. 10F shows that L-NAME (50 μM) significantly reduced the BH4 mediated increase in [Ca²⁺]_{I} but has no effect on the DEA-NONOate (NO-donor (50 μM)) induced increase of [Ca²⁺]_{I}. For all panels, asterisks (*) indicates a p<0.05.

FIG. 11A shows the physical locations of the fifteen genotyped single nucleotide polymorphisms (SNPs) and haplotype analysis for the GTP cyclohydrolase gene (GCH1). Coding exons are shown as blocks. SNP locations are from SNP browser software and the Panther Classification System public database, August 2005 or the Ensemble database v.38, April 2006. P values for significant SNPs are shown for the primary outcome of leg pain over the 12 months following lumbar discectomy surgery. Those significantly associated with low pain scores are indicated by a star (*p<0.05; pain scores for each SNP). The letters in each haplotype are the genotypes for the 15 SNPs in GCH1. Only haplotypes with frequency >1% are included. Eight haplotypes account for 94% of the chromosomes studied. Pain scores for each haplotype are the mean Z-score for “leg pain” over the year after lumbar discectomy, adjusted for covariates, and weighted for the probability in each patient that the algorithm-based assembly of two haplotypes from the patient's SNP assays was correct. Lower scores correspond to less pain. The score was calculated from four questions assessing frequency of pain at rest, after walking, and their improvement after surgery. Haplotype ACGTTGCACACGAGG (highlighted in white) has a lower pain score for “leg pain” than the seven other haplotypes. p 0.009.

FIG. 11B is a chart showing the effect of the number of copies of the pain protective haplotype on pain scores. There is a roughly linear reduction in persistent pain associated with the number of copies of the haplotype ACGTTGCACACGAGG, with the caveat that only four patients were homozygous for this haplotype.

FIGS. 12A and 12B show SPR and QDRP gene structures, respectively, and SNP mapping. Coding exons are shown as solid blocks. Physical locations are from the National Center for Biotechnology Information (NCBI) database and SNP Browser Program (ABI), August 2005. P values for each SNP shown for the primary outcome of “leg pain” over one year following lumbar discectomy surgery.

FIGS. 13A-13C show haplotype block organization of GCH1 (FIG. 13A), SPR (FIG. 13B), and QDPR (FIG. 13C). Each box represents the percentage linkage disequilibrium, D′ (% LD) between pairs of SNPs, as generated by Haploview software (Whitehead Institute for Biomedical Research, USA). D′ is color coded, with a dark box indicating complete linkage disequilibrium (D′=1.00) between locus pairs. GCH1 and SPR each have a single haplotype block spanning the entire gene, with some disruption of linkage disequilibrium in GCH1 due to low allelic frequency of several markers. QDPR has two haploblocks. FIG. 13A also shows GCH1 haplotypes were identified in-silico using PHASE software, which implements a modified Expectation/Maximization (EM) algorithm to reconstruct haplotypes from population genotype data. A further analysis assessed linkage disequilibrium between SNPs describing the non-independence of alleles. Linkage disequilibrium was quantified as D=p_{AB}−P_{A}·p_{A}, where D is a measure of linkage disequilibrium between cDNA positions A and B. P_{AB} denotes the frequency of sequences that contain allele A at the first position and allele B at the second position, and p_{A} and p_{B} are the frequencies of the respective alleles. Because “D” depends on the allelic frequency, D was normalized to its theoretical maximum, resulting in a value of D′ which ranges between 0 and 1 for complete linkage equilibrium and disequilibrium, respectively. Linkage disequilibrium was additionally quantified by r² denoting the squared correlation between the two loci. Each box represents the linkage disequilibrium, D′ between pairs of SNPs, as generated by HelixTree® software. D′ is grey-scale coded, with a white box indicating complete linkage disequilibrium (D′=1.00) between locus pairs. GCH1 has a single haplotype block spanning the entire gene, with some disruption of linkage disequilibrium in GCH1 due to low allelic frequency of several markers.

FIGS. 14A and 14B show the effects of copy number of the pain protective haplotype in various tests. FIG. 14A shows the effect of number of copies of the pain protective haplotype on frequency of leg pain at rest. 0/0, X/0, and X/X denote patients with zero, one, and two copies of haplotype, respectively. Numbers on y-axis correspond to pain frequency: always (6), almost always (5), usually (4), about half the time (3), a few times (2), rarely (1), and not at all (0). FIG. 14B shows the effect of number of copies of pain protective haplotype (0/0 n=384; X/0 n=153; and X/X n=10) on experimental pain sensitivity in healthy volunteers (**p<0.01 compared with 0/0 group).

FIGS. 14C-14F show the effect of forskolin on patient white blood cells. FIG. 14C shows GCH1 mRNA (QRT-PCR) in EBV immortalized WBCs of 0/0 (n=7), X/0 (n=5) and X/X (n=4) lumbar root pain patients, stimulated with forskolin (10 μM, 12 hrs), relative to unstimulated levels in 0/0 individuals (100%). White bars unstimulated; grey after stimulation. FIG. 14D shows GCH1 protein expression in immortalized WBCs and % change after forskolin treatment. FIG. 14E shows biopterin in supernatants of forskolin stimulated immortalized WBCs, and FIG. 14F shows forskolin (10 μM, 24 h) stimulated whole blood from healthy volunteers (0/0 n=11; X/X n=10) relative to baseline. Results represent means with SEM. Linear regression analysis revealed significant effects of number of copies of pain protective haplotype for forskolin induced changes in GCH1 mRNA (p<0.001), protein (p=0.037) and biopterin (p=0.001 and p=0.002).

FIG. 15 shows the effect of the number of copies of a putative “pain protective haplotype” on experimental pain sensitivity. The graph shows temporal summation responses to repeated heat stimuli. Each value represents the mean ±standard error of the verbal numerical magnitude estimate obtained for each thermal (53° C.) pulse. Non painful warn sensations were rated between 0-19. Thermal stimuli, that evoked heat pain sensations were rated between 20 (pain threshold) and 100 (most intense pain imaginable). Each value represents the mean with associated s.e.m. The association of the number of copies of the “pain protective haplotype” with the temporal summation of heat pain was analyzed using a one-way ANOVA followed by Bonferroni adjustment for post-hoc testing (p<0.001 for groups 0/0 and X/0 vs. group X/X comparison).

FIGS. 16A-16C show the downregulation of KCNS1 in the SNI, CCI, and SNL models of peripheral neuropathic pain, as detected by Affymetrix RGU34A microarrays (n=3, error SEM). Asterisks (*) indicate a p<0.05.

FIGS. 17A-17C show in situ hybridization for KCNS1 mRNA within the rat DRG. The KCNS1 mRNA signal is shown in the naïve DRG (FIG. 17A), in DRG 7 days post SNI (FIG. 17B), and 7 days post CCI (FIG. 17C). Downregulation is evident in large diameter cells (scale 100 μm).

FIG. 18 shows the location of mutations identified in the genomic region of the KCNS1 gene, including SNP mapping.

FIG. 19 shows haplotype block organization of the KCNS1 gene. Details regarding the block diagram is described above, in the description of FIGS. 13A-13C.

### DETAILED DESCRIPTION

The present invention features methods for diagnosing patients with an altered sensitivity to pain, an altered susceptibility to developing acute or chronic pain, based on the identification of haplotypes in two genes, GCH1 and KCNS1, or a propensity to develop a BH4-related disorder, based on haplotypes in GCH1. These haplotypes can be diagnostic of pain sensitivity, acute or persistent pain development, or abnormal pain amplification. GCH1, a gene encoding a key enzyme in BH4 synthesis, was identified from a group of three genes whose transcripts are upregulated in response to peripheral nerve injury. The presence of a GCH1 haplotype was found to be protective against persistent radicular pain after surgical diskectomy and associated with reduced sensitivity to experimental pain. In addition, we observed that white blood cells from individuals with the pain protective GCH1 haplotype exhibited decreased GCH1 expression and activity upon forskolin challenge, thus demonstrating that the haplotype is functionally significant. Constitutive levels of GCH1 were normal in individuals with the pain protective GCH1 haplotype but the induction of GCH1 mRNA, protein and activity in response to a challenge, was reduced. On this basis, we believe this haplotype may be associated with an altered (e.g., increased or decreased) risk of developing a BH4-related disorder, for example, a disease involving endothelial cell function or a cardiovascular system disease (e.g., ischemic reperfusion injury, cardiac hypertrophy, vasculitis, and systemic and pulmonary hypertension) or a nervous system disease.

A second gene KCNS1 was likewise identified as possessing haplotype markers that correlate with pain sensitivity and chronic pain and that can therefore also be used as diagnostic markers according to the invention. These genes were identified by searching, using microarrays, both for genes regulated over time (3 to 40 days) in the rat DRG in three models of peripheral neuropathic pain: the spared nerve injury (SNI), chronic constriction injury (CCI), and spinal nerve ligation model (SNL) and for those that belong to common metabolic, signaling, or biosynthetic pathways. Transcripts for two of the three enzymes in the BH4 synthetic pathway, GCH1 and SR, were found to be upregulated in these models as was the BH4 recycling enzyme QDPR. Another gene identified with this screen was the potassium channel KCNS1, which was down-regulated in DRG all three models of peripheral neuropathic pain.

### EXAMPLE 1

### GCH1 Pain Protective Haplotypes

Involvement of BH4 Synthesis in Pain

Enzymes that synthesize or recycle the enzyme cofactor BH4, as described below, are upregulated in sensory neurons in response to peripheral nerve injury, and this pathway is also activated by peripheral inflammation. Blocking BH4 synthesis by independently inhibiting two of its synthesizing enzymes reduces acute and established neuropathic pain and prevents or diminishes inflammatory pain. Conversely, BH4 administration produces pain in naïve animals and enhances pain sensitivity in animals with either nerve injury or inflammation. Thus, BH4 synthesizing enzymes may be major regulators of pain sensitivity and BH4 may be an intrinsic pain-producing factor.

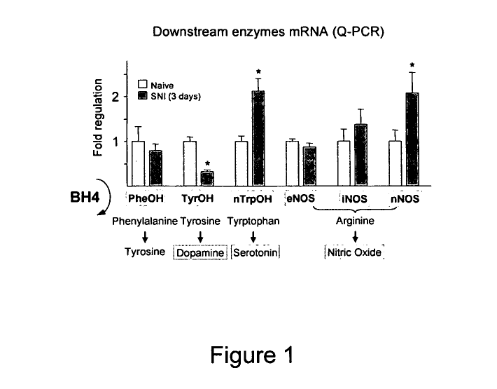

BH4 is an essential cofactor for several major enzymes; no reaction occurs in its absence even in the presence of substrate. BH4 levels therefore need to be tightly regulated. The absence or substantial reduction of BH4 production due to a loss-of-function mutation in the coding region of GTP cyclohydrolase or sepiapterin reductase genes results in severe neurological problems from a decrease or absence of amine transmitters (Segawa et al., Ann Neurol 54(Suppl 6):S32-45 (2003); Neville et al., Brain 128:2291-2296 (2005)). Elevation of BH4 levels, by increasing amine and nitric oxide synthesis may also be deleterious, particularly if downstream enzymes are also upregulated. Three days following nerve injury, an upregulation of neuronal tryptophan hydroxylase and neuronal nitric oxide synthase in ipsilateral DRGs occurs, supporting results of previous studies (FIG. 1; Luo et al., J Neurosci 19:9201-9208 (1999)) and suggesting that overproduction of serotonin and nitric oxide might mediate the pain evoked by BH4. Under physiologic conditions, BH4 negatively regulates its production by binding to GTP cyclohydrolase feedback protein (GFRP) which inhibits GTP cyclohydrolase activity. GFRP, unlike GTP cyclohydrolase, is not upregulated after nerve injury (data not shown). BH4, when present in stoichiometric excess of GFRP, does not exert efficient feedback inhibition on GTP cyclohydrolase. The resulting accumulation of an excess of BH4 in DRG neurons can then induce or enhance pain sensitivity.

Elevated BH4 levels may cause BH4-dependent enzymes expressed in DRG neurons to be activated, may cause BH4 to be released from the neurons (Choi et al., Mol Pharmacol 58:633-40 (2000)) which may then act on neighboring cells (e.g., neuronal or non-neuronal cells) to regulate their enzymatic activity, or may exert a cofactor-independent action (Koshimura et al., J Neurochem 63:649-654 (1994); Mataga et al., Brain Res 551:64-71 (1991); Ohue et al., Brain Res 607:255-260 (1993)). A direct effect of BH4 on the excitability or synaptic efficacy of dorsal horn neurons was not observed. Because BH4 produces pain rapidly (<30 min), the pain-related effects likely do not involve long latency changes such as altered transcription, activation of microglia (Tsuda et al., Trends Neurosci 28:101-107 (2005)), or induction of neuronal cell death (Scholz et al., J Neurosci 25:7317-7323 (2005)). Similarly, as the GTP-cyclohydrolase inhibitor DAHP has a rapid onset of analgesic action and continues to be effective upon repeated administration (see below), a continued excess presence of BH4 may be required for its role in chronic pain. The efficacy of DAHP in the formalin test, peripheral inflammation, and multiple models of neuropathic pain, as described below, indicates a mechanism common to these diverse models. One possibility is the use-dependent central sensitization of dorsal horn neurons (Woolf, C. J., Nature 306:686-688 (1983)), which is common to the formalin, inflammatory, and neuropathic pain models. The effect of the “pain protective” GCH1 haplotype described below on pain arising from repeated heat pain stimulation, supports this idea, as this experimental pain model in humans appears to be contributed to by central changes in excitability (Price et al., Pain 59:165-174 (1994); Eide, P. K., Eur J Pain 4:5-15 (2000); Maixner et al., Pain 76:71-81 (1998); Vierck et al., J Neurophysiol 78:992-1002 (1997)). Nevertheless, DAHP also acts in phase one of the formalin test, and the GCH1 haplotype alters the immediate response to a noxious stimulus in humans. Thus, BH4 appears to contribute to the sensitivity to acute nociceptive stimuli.

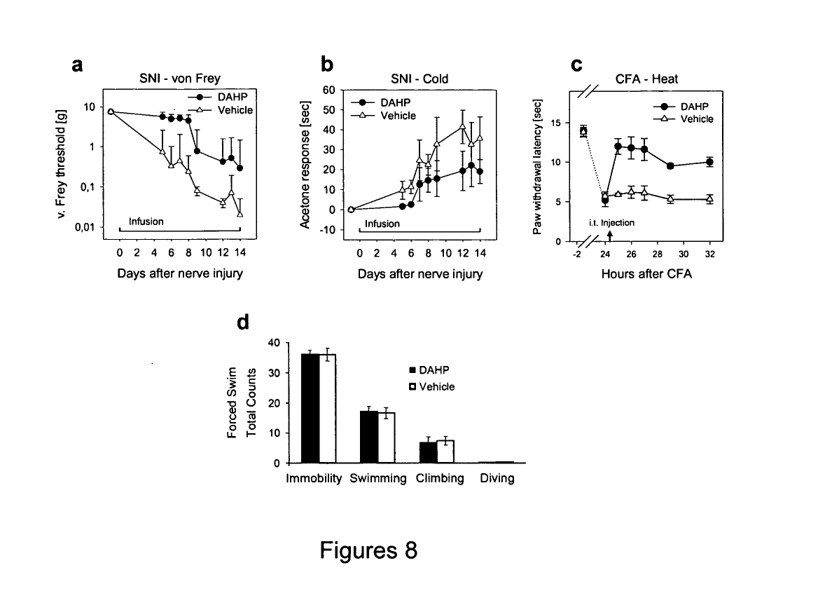

Seven days after SNI, nitric oxide levels increase in the DRG, suggesting that NO overproduction contributes to the pain evoked by BH4. Pain producing effects of NO probably involve direct nitrosylation of target proteins (Hara et al., Nat Cell Biol 7:665-674 (2005)), modulation of NMDA receptor activity (Lipton et al., Nature 364:626-632 (1993)), and/or activation of the guanylyl cyclase-cGMP-PKG pathway (Tegeder et al., Proc Natl Acad Sci USA 101:3253-3257 (2004); Lewin et al., Nat Neurosci 2:18-23 (1999)) resulting in increased glutamatergic transmission (Huang et al., Mol Pharmacol 64:521-532 (2003)). Supporting this, inhibition of GTP cyclohydrolase prevents increases in both BH4 and NO, and NOS inhibition reduces mechanical and cold allodynia after SNI. BH4 may act in a paracrine as well as an autocrine fashion, as it is released from neurons (Choi et al., Mol Pharmacol 58:633-640 (2000)) and may both increase enzyme activity and produce cofactor-independent effects (Koshimura et al., J Neurochem 63:649-654 (1994); Shiraki et al., Biochem Biophys Res Commun 221:181-185 (1996)). Considering the latter, we found that BH4 produces a short latency calcium influx in cultured adult DRG neurons partly mediated through nitric oxide synthesis. Although neuronal tryptophan hydroxylase mRNA was upregulated in DRG neurons after SNI serotonin levels remained below detection limits in this tissue. In the spinal cord serotonin is expressed in descending inhibitory and excitatory fibers. DAHP treatment did not, however, significantly reduce serotonin concentrations in the spinal cord and brain stem (data not shown) or alter the forced water swim test (see FIG. 8D and described below). This model of anxiety and depressive behavior is sensitive to changes in serotonin levels (Mague et al., J Pharmacol Exp Ther 305:323-330 (2003)). Thus, we believe that changes in serotonin production do not contribute to BH4-mediated increases in pain sensitivity. Because BH4 produces pain rapidly, these immediate effects likely do not involve transcriptional changes, activation of microglia (Tsuda et al., Trends Neurosci 28:101-107 (2005)), or induction of neuronal cell death (Scholz et al., J Neurosci 25:7317-7323 (2005)). Moreover, the efficacy of DAHP in the formalin test, peripheral inflammation, and multiple models of neuropathic pain, points to a common BH4-dependent mechanism in diverse pain conditions.

To evaluate the potential role of BH4 in human pain, we analyzed whether polymorphisms in GCH1, the rate-limiting BH4 synthesizing enzyme, are associated with specific pain phenotypes. If BH4 is absent or substantially reduced in humans due to rare missense, nonsense, deletion, or insertion mutations in the coding regions of GTP cyclohydrolase (Hagenah et al., Neurology 64:908-911 (2005)) or sepiapterin reductase genes, dopa-responsive dystonia and other severe neurological problems occur due to absence of amine transmitters (Ichinose et al., Nat Genet 8:236-242 (1994); Bonafe et al., Am J Hum Genet 69:269-277 (2001)). It is not known whether pain perception is affected by these rare mutations. Our homozygotes for the pain protective haplotype did not have any neurological diseases. We therefore speculated that the pain protective haplotype embodies a variation in a regulatory site that causes a modest impairment in GTP cyclohydrolase production or function. In support of this, constitutive expression of GTP cyclohydrolase and BH4 production was found to be equivalent in cells of carriers and non-carriers of the pain protective haplotype. However, forskolin-evoked upregulation was significantly reduced in carriers of the pain protective haplotype. Thus, we believe that the locations mediating GCH1 transcription involve elements in the region 5′ to exon-1 and within the large 20 kb intron-1 because the SNPs exclusively found in the pain protective haplotype are located in the putative promoter region of GCH1 (C.-9610G>A) and in intron-1 (C.343+8900A>T), respectively. These SNPs may modify transcription efficiency to signals mediated by cAMP-dependent transcription factors. Although hundreds of transcripts are regulated in DRGs by nerve injury or sustained nociceptor stimulation, and although many chemical agents and biologic molecules affect pain behavior in experimental settings, only few genes have been identified so far that modulate pain sensitivity in humans (Zubieta et al., Science 299:1240-1243 (2003); Mogil et al., Proc Natl Acad Sci USA 100:4867-4872 (2003)). The current finding for GCH1 is one of the first to be replicated across human populations.

Here, alterations in the level of the essential enzyme cofactor BH4 modify the sensitivity of the pain system, and single nucleotide polymorphisms in the gene for the rate-limiting BH4-producing enzyme GTP cyclohydrolase alter both responses in healthy humans to noxious stimuli and the susceptibility of patients for developing persistent neuropathic pain. Because the pain protective haplotype in GCH1 is associated with a reduction in the risk of developing persistent pain without signs of dystonia, a treatment strategy that could reduce excess de novo BH4 synthesis in the DRG, but not constitutive BH4 by targeting only induction of GTP cyclohydrolase or by leaving the recycling pathway intact, may provide a means for preventing the establishment or maintenance of chronic pain. Further, identification of a predictor of the intensity and chronicity of pain is a useful tool to assess an individual patient's risk for developing chronic pain. The effect of the pain protective haplotype on both experimental and persistent pain, and the involvement of BH4 in both inflammatory and neuropathic pain, may explain why sensitivity to acute experimental pain is a predictor of postsurgical and eventually chronic pain (Bisgaard et al., Pain 90:261-269 (2001); Bisgaard et al., Scand J Gastroenterol 40:1358-1364 (2005)).

Identification of the Link Between BH4 Synthesis and Chronic Pain

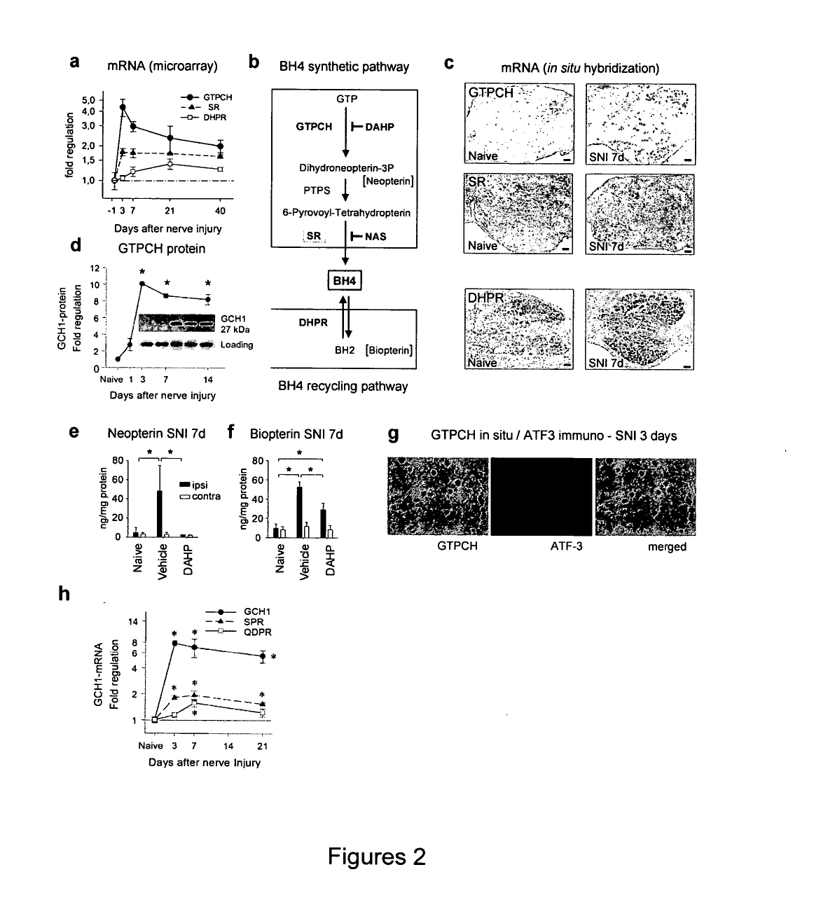

The link between BH4 synthesis and chronic pain was identified by searching the several hundred genes regulated in the dorsal root ganglion (DRG) following sciatic nerve injury for genes belonging to common metabolic, signaling, or biosynthetic pathways (Costigan et al., BMC Neurosci 3:16 (2002)). These genes are involved in producing chronic neuropathic pain. The regulated enzymes are GTP cyclohydrolase, which catalyzes the first, rate-limiting step, and sepiapterin reductase, which performs the final conversion of 6-pyrovoyl-tetrahydropterin to tetrahydrobiopterin (FIGS. 2A-2G).

BH4 is an essential cofactor for phenylalanine, tyrosine, and tryptophan hydroxylase and for nitric oxide synthases. Its availability, along with enzyme and substrate levels, is critical for catecholamine, serotonin, and nitric oxide synthesis and phenylalanine metabolism (Kobayashi et al., J Pharmacol Exp Ther 256:773-9 (1991); Khoo et al., Circulation (2005); Cho et al., J Neurosci 19:878-89 (1999); Thony et al., Biochem J 347(Pt 1):1-16 (2000)). Mutations in GTP cyclohydrolase or sepiapterin reductase that cause a congenital BH4 deficiency in the brain are characterized by symptoms related to monoamine neurotransmitter deficiency, resulting in dopa-responsive motor, psychiatric, and cognitive disorders (Segawa et al., Ann Neurol 54(Suppl 6):S32-45 (2003); Neville et al., Brain 28(Pt 10):2291-2296 (2005)). The production of BH4 is tightly regulated by GTP cyclohydrolase transcription and activity (Frank et al., J Invest Dermatol 111: 1058-1064 (1998); Bauer et al., J Neurochem 82:1300-1310 (2002)). Phosphorylation (Hesslinger et al., J Biol Chem 273:21616-21622 (1998)), feed-forward activation through phenylalanine (Maita et al., Proc Natl Acad Sci USA 99:1212-1217 (2002)), and feedback inhibition through BH4, both acting in concert with a GTP cyclohydrolase feedback regulatory protein (GFRP) (Maita et al., J Biol Chem 279:51534-51540 (2004)), all regulate GTP cyclohydrolase activity. Mutations in GTP cyclohydrolase or sepiapterin reductase that cause monoamine neurotransmitter deficiency, result in dopa-responsive motor, psychiatric and cognitive disorders (Ichinose et al., Nat Genet 8:236-242 (1994); Bonafe et al., Am J Hum Genet 69:269-277 (2001)). Given the absolute requirement for this cofactor for monoamine and nitric oxide synthesis, and the vital roles of these neurotransmitters in the nervous system, increasing BH4 levels may have a profound impact on neuronal signaling. As described herein, BH4 levels are critical for neuropathic and inflammatory pain, and a genetic polymorphism of GTP cyclohydrolase is associated with reduced pain sensitivity and chronicity in humans due to reduced BH4 production.

Upregulation of Tetrahydrobiopterin Synthesizing Enzymes

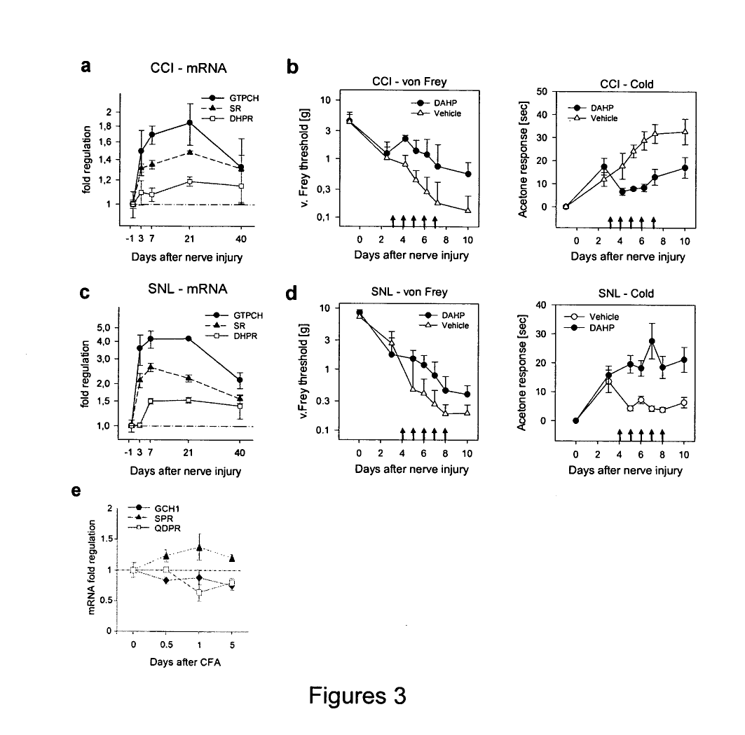

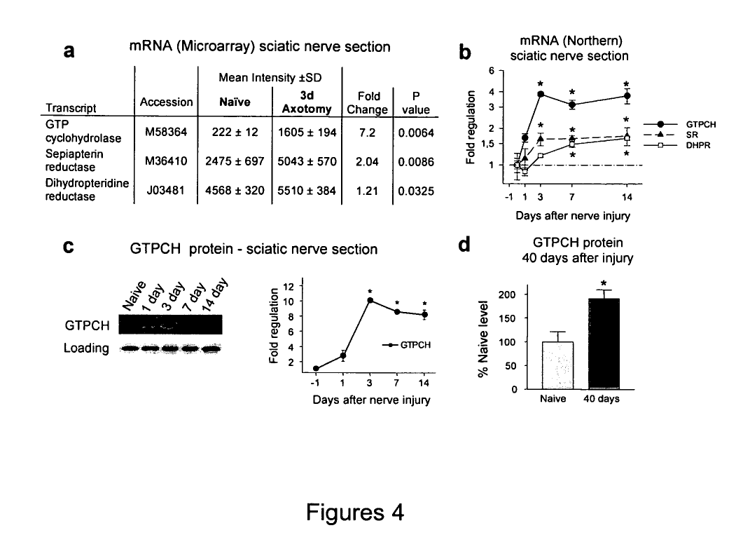

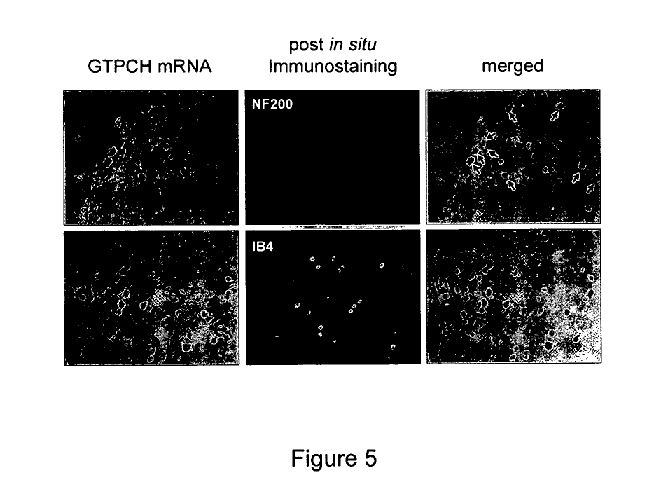

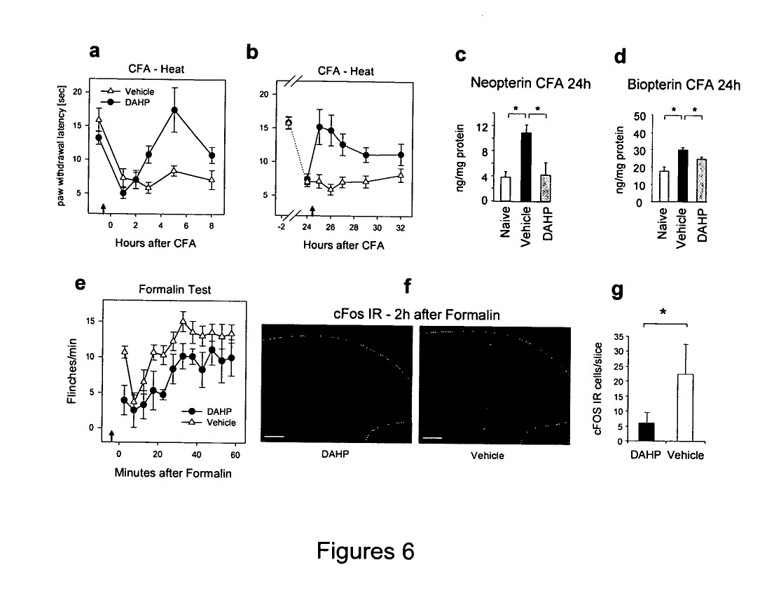

The expression of GTP cyclohydrolase and sepiapterin reductase over time in L4/5 DRGs was studied in three models of peripheral neuropathic pain: (i) the spared nerve injury (SNI) (Decosterd and Woolf, Pain 87:149-58 (2000)), (ii) chronic constriction injury (CCI) (Bennett and Xie, Pain 33:87-107 (1988)), and (iii) spinal nerve ligation model (SNL) (Kim and Chung, Pain 50:355-63 (1992)). In addition, expression in the intraplantar complete Freund's adjuvant (CFA) paw inflammation model was studied. These models produce long lasting heightened pain sensitivity including mechanical and cold allodynia as well as mechanical and heat hyperalgesia. GTP cyclohydrolase and sepiapterin reductase transcripts were upregulated in lumbar (L4/5) DRGs in all three nerve injury models (SNI FIG. 2A, CCI and SNL FIGS. 3A-3D), and sepiapterin reductase mRNA was also increased in DRGs after CFA-induced paw inflammation (FIG. 3E). Further, after nerve injury a modest upregulation of dihydropteridine reductase (DHPR), the enzyme that recycles BH4 from its oxidation products biopterin and dihydrobiopterin, was observed. The upregulation of the transcripts of the three enzymes in DRG neurons was confirmed by in situ hybridization in the SNI model (FIG. 2C). The induction of GTP cyclohydrolase mRNA was accompanied by increased protein expression (FIG. 2D; FIGS. 4A-4G) and activity (FIG. 2E), as indicated by increased levels of neopterin, an inactive metabolite of the first intermediate product in the synthesis cascade, dihydroneopterin-triphosphate (Rebelo et al., J Mol Biol 326:503-516 (2003)) (FIG. 2E). A shift to neopterin normally prevents accumulation of the intermediate and overproduction of the end product BH4. Following nerve injury, however, the upregulation and activation of the pathway caused a marked increase in BH4 levels, as indicated by the increase in its stable oxidation product, biopterin (FIG. 2F). Combined in situ hybridization and immunostaining of GTP cyclohydrolase mRNA and the injury-induced nuclear transcription factor ATF-3 (Tsujino et al., Mol Cell Neurosci 15:170-182 (2000)) showed that GTP cyclohydrolase is upregulated only in injured neurons (FIG. 2G) with myelinated and unmyelinated axons (FIG. 5). In particular, double labeling of GTP cyclohydrolase mRNA and the injury-induced nuclear transcription factor ATF-3 (Tsujino et al., Mol Cell Neurosci 15:170-182 (2000)) revealed that 97±3% of neurons upregulating GTP cyclohydrolase are ATF-3 positive (FIG. 2G). Seven days after SNI 65±13% of L5 DRG neuronal nuclei express ATF-3, reflecting the proportion of cells with axon damage (Decosterd et al., Pain 87:149-158 (2000)). Of these, 75±4% upregulate GTP cyclohydrolase mRNA. Although not upregulated after CFA, GTP cyclohydrolase activity and BH4 production were increased in DRGs in CFA-induced paw inflammation (FIGS. 6C and 6D), albeit to a lesser extent than after nerve injury.

Inhibition of Neuropathic and Inflammatory Pain by Blocking BH4 Synthesis

To test if the observed increase in BH4 synthesis contributes to neuropathic and inflammatory pain, the effects of inhibitors of BH4-synthesizing enzymes in three models of peripheral neuropathic pain and in CFA-induced paw inflammation were analyzed. 2,4-diamino-6-hydroxypyrimidine (DAHP), the prototypic GTP cyclohydrolase inhibitor, was used to block GTP cyclohydrolase activity (Kolinsky and Gross, J Biol Chem 279:40677-40682 (2004); Yoneyama et al., Arch Biochem Biophys 388:67-73 (2001); Xie et al., J Biol Chem 273:21091-21098 (1998)). DAHP, like BH4, specifically binds at the interface of GTP cyclohydrolase and its feedback regulatory protein GFRP to form an inhibitory complex that blocks GTP cyclohydrolase activity (Maita et al., J Biol Chem 279:51534-51540 (2004)). DHAP is a low potency but specific inhibitor. Minor modifications of DAHP cause it to lose this inhibitory activity (Yoneyama et al., Arch Biochem Biophys 388:67-73 (2001)) and prevent DAHP from directly interacting with any of the BH4-dependent enzymes.

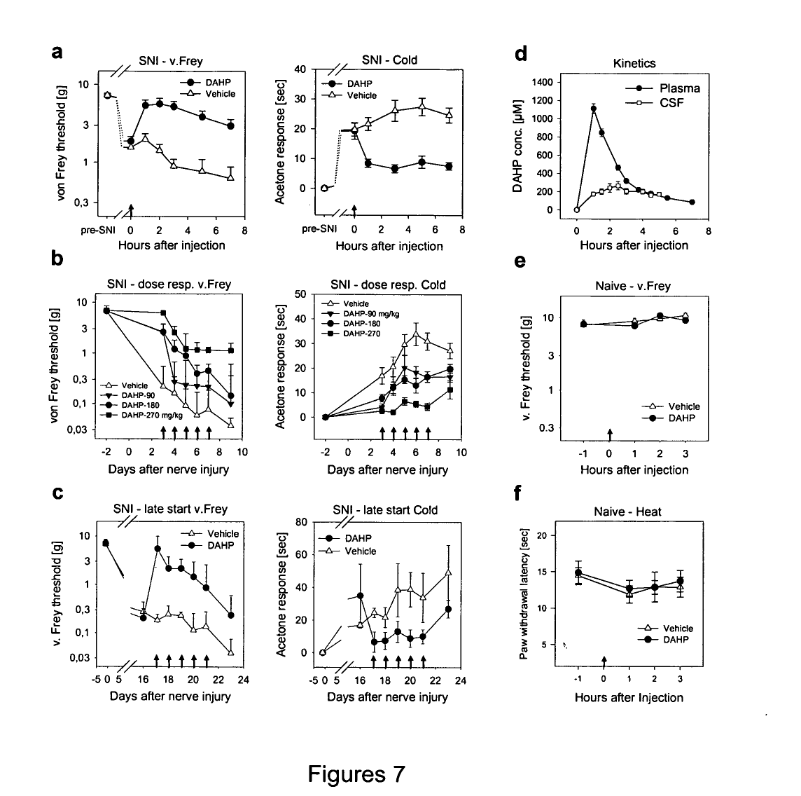

Injection of a single dose of DAHP (180 mg/kg i.p.) four days after sciatic nerve injury (SNI model), a time when pain hypersensitivity is present, reverses mechanical and cold pain hypersensitivity within 60 minutes (FIG. 7A). The antinociceptive effect of DAHP parallels the time course of its plasma and CSF concentrations (FIG. 7D), which are within the IC₅₀ range (100-300 μM) for GTP cyclohydrolase inhibition determined in vitro (Kolinsky and Gross, J Biol Chem 279:40677-40682 (2004); Xie et al., J Biol Chem 273:21091-21098 (1998)). DAHP treatment at this dose completely prevents the nerve injury induced increases in neopterin (FIG. 2E), and significantly reduces biopterin levels (FIG. 2F) in injured DRGs. Biopterin levels did not return to pre-injury baseline after DAHP treatment because the recycling of BH4 from its oxidation products is not inhibited by DAHP. Nevertheless inhibiting de novo synthesis of BH4 and decreasing the BH4 excess significantly reduces neuropathic pain (FIGS. 7A-7C). The relative efficacy of DAHP, measured as the extent of return to pre-surgery baseline values, exceeds that of non-sedating doses of morphine, gabapentin, amitriptyline, and carbamazepine that we have measured in the SNI model (Decosterd et al., Anesth Analg 99:457-463 (2004)). DAHP produces dose-dependent reductions in mechanical and cold allodynia in all three neuropathic pain models (FIG. 7A-7C for SNI, FIGS. 3B and 3D for CCI and SNL). Likewise, intrathecal DAHP (250 μg/kg/h; 1/30^{th} of the systemic dose) reduces mechanical and cold allodynia after SNI (FIGS. 8A-8C). Further, DAHP decreases pain hypersensitivity when first administered seventeen days after SNI surgery, when pain hypersensitivity has been established for more than two weeks (FIG. 7C). Repeated daily administration of DAHP continues to produce analgesia without obvious loss of activity (FIGS. 7B and 7C). No deleterious effect of acute single or daily treatment on general well-being, body weight, gait, or activity was observed. This indicates that a reduction in elevated BH4 levels can reduce pain without producing abnormal neurological function.

DAHP (180 mg/kg i.p.) did not change the mechanical threshold for paw withdrawal or radiant heat evoked paw withdrawal latency in naïve animals (FIGS. 7E and 7F) and had no effect on body weight, activity, or performance in the forced swim test (FIG. 8D). Inflammation produced by hindpaw injection of CFA did not increase GTP cyclohydrolase mRNA expression in the DRG (FIG. 3E). However, intraplantar CFA caused significant increases in GTP cyclohydrolase enzyme activity, with increases of neopterin (FIG. 6C) and biopterin (FIG. 6D) in L4/5 DRGs. The treatment did, however, reduce CFA-evoked heat hyperalgesia of the inflamed hindpaw (FIGS. 6A and 6B), both when administered before the onset of inflammation (FIG. 6A) and 24 hours after interplantar CFA injection (FIG. 6B), and normalized neopterin and biopterin levels in the DRGs (FIGS. 6C and 6D). Similar efficacy is achieved with intrathecal DAHP (FIGS. 8A-8C; 1/30^{th} of the systemic dose). DAHP administration completely prevents the inflammation-evoked increase of neopterin and significantly reduces elevated biopterin levels in ipsilateral L4/L5 DRGs (FIGS. 6C and 6D). DAHP (180 mg/kg i.p.) treatment also significantly reduces the flinching behavior in the first and second phases of the formalin test, which are indicative of acute nociception and activity-dependent central sensitization in the spinal cord, respectively (FIG. 6E). This antinociceptive effect is accompanied by a significant reduction in the number of cFos immunoreactive neurons in the ipsilateral dorsal horn of the spinal cord found two hours after formalin injection (FIGS. 6F and 6G). c-Fos induction in dorsal horn neurons is a useful surrogate marker of nociceptive synaptic processing, and this finding indicates that reducing BH4 levels reduces synaptic transmission at the first elements in the central pain pathways.

Inhibition of Pain by Blocking Sepiapterin Reductase

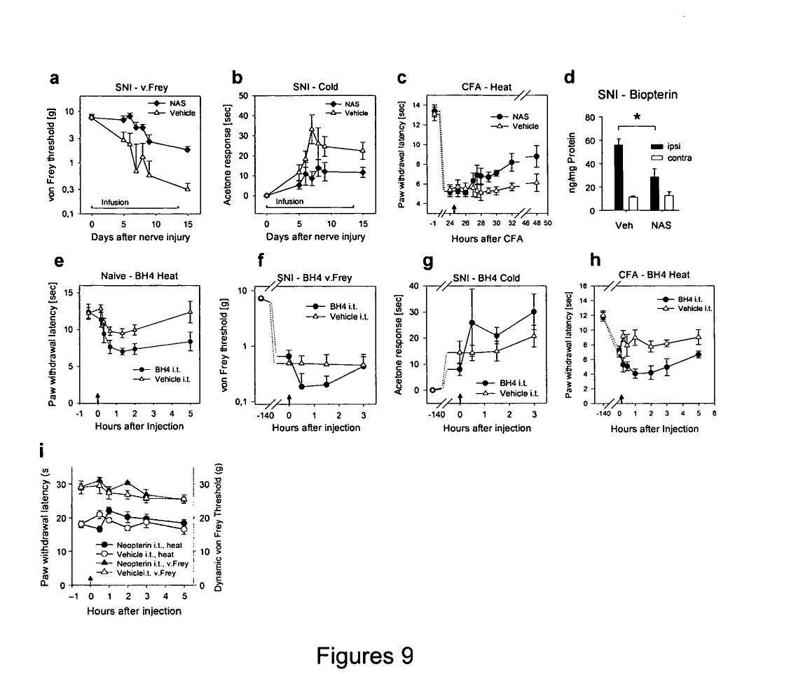

To substantiate that the analgesic effects of DAHP result from reduced BH4 synthesis, the effect of N-acetyl-serotonin (NAS), an inhibitor of sepiapterin reductase, was also tested (Milstien and Kaufman, Biochem Biophys Res Commun 115:888-893 (1983)). NAS (100 μg/kg/hr) significantly reduces nerve-injury evoked mechanical and cold allodynia (FIGS. 9A and 9B) after SNI without overt adverse effects. Intraperitoneal injection of a single dose of NAS (50 mg/kg i.p.) before induction of paw inflammation significantly reduces thermal hyperalgesia in the CFA paw inflammation model (FIG. 9C). NAS also significantly reduces total biopterin levels in L4/5 DRGs after SNI, indicating inhibition of BH4 synthesis (FIG. 9D).

Induction of Pain Hypersensitivity by Tetrahydrobiopterin

To determine if BH4 enhances pain sensitivity in naïve animals, we injected its active enantiomer, (6R)-5,6,7,8-tetrahydrobiopterin dihydrochloride, intrathecally (1 μg/μl, 10 μl). 6R-BH4 causes a prompt and long lasting increase in response to noxious radiant heat (FIG. 9E). Intrathecally injected BH4 also further increases pain sensitivity after both SNI evoked nerve injury (FIGS. 9F and 9G). BH4 further increased heat pain sensitivity when injected intrathecally 5 days after CFA (FIG. 9H). This indicates that overproduction of BH4 heightens pain sensitivity. However, 6R-BH4 bath-applied to an isolated adult rat spinal cord slice does not produce a change in the frequency or amplitude of AMPA receptor mediated miniature excitatory postsynaptic currents or direct inward currents of superficial dorsal horn neurons (6R-BH4 10 μM n=6; 20 μM n=2; data not shown) indicating that it does not increase glutamate release or responsiveness. Intrathecal administration of the inactive metabolite neopterin (1 μg/μl, 10 μl i.t.) had no significant effect (FIG. 9I).

Potential Mechanisms

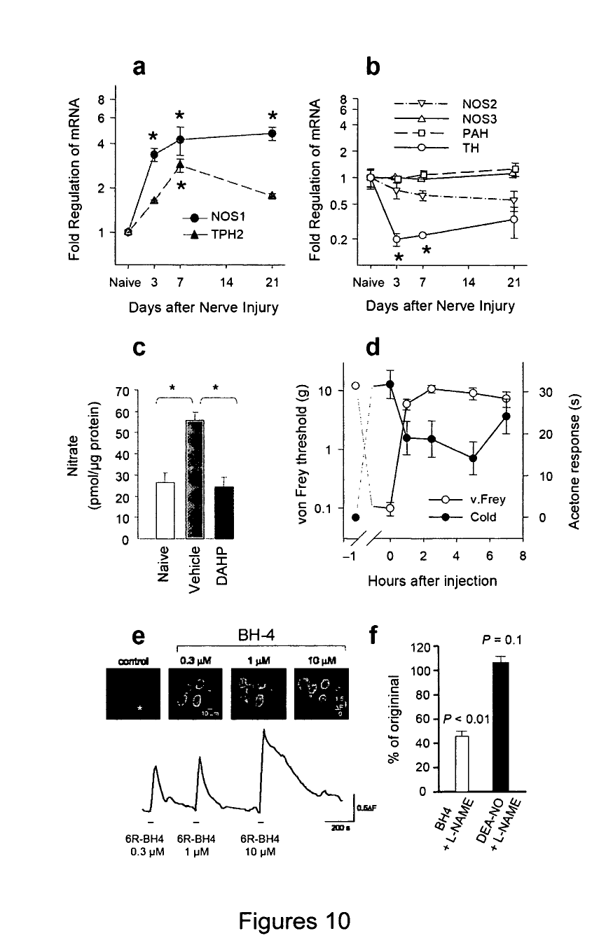

Availability of BH4 regulates activity of NO synthases as well as tyrosine and tryptophan hydroxylases. Therefore, its pain producing effects may be mediated through excess activity of these enzymes. Following SNI, neuronal tryptophan hydroxylase and neuronal nitric oxide synthase (nNOS) in ipsilateral DRGs are upregulated (FIG. 10A), but there is no change in phenylalanine hydroxylase, endothelial or inducible NOS, or a decrease in tyrosine hydroxylase (FIG. 10B). Despite upregulation of neuronal tryptophan hydroxylase in the DRG, serotonin levels in DRGs from naïve and SNI animals were below limits of quantification (data not shown). Upregulation of nNOS was accompanied by an increase in nitric oxide levels in the L4/5 DRGs at day seven (FIG. 10C) that was prevented by DAHP treatment. The NOS inhibitor L-NAME (25 mg/kg i.p.) reduced SNI-evoked mechanical and cold allodynia tested four days after SNI (FIG. 10D). Antinociceptive effects of DAHP may be mediated at least in part, therefore, by preventing excess NO production.

To further analyze potential mechanisms, we employed calcium imaging with cultured adult rat DRG neurons. 6R-BH4 (0.3-10 μM) dose-dependently increased intracellular calcium levels in 67% of recorded cells (n=95; FIG. 10E). BH4 elevated calcium within seconds, and this was abolished by a calcium-free perfusate, indicating increased calcium influx (n=12). The NO releasing substance DEA-NONOate (50 μM) produced similar increases in [Ca²⁺]₁, which were also mediated by calcium influx (n=32). The NOS inhibitor L-NAME reduced the BH4 effect by 47±4% (n=29, p<0.01; FIG. 10F) suggesting that BH4 acts partly but not exclusively through NOS.

Bath-applied 6R-BH4 to an isolated adult rat spinal cord slice did not change the frequency or amplitude of AMPA receptor mediated miniature excitatory postsynaptic currents or produced direct inward currents in superficial dorsal horn neurons (6R-BH4 10 μM n=6; 20 μM n=2; data not shown) indicating that BH4, in contrast to nitric oxide (Pan et al., Proc Natl Acad Sci USA 93:15423-15428 (1996)), does not increase glutamatergic transmission.

Pain Protective Haplotype of GTP Cyclohydrolase in Humans

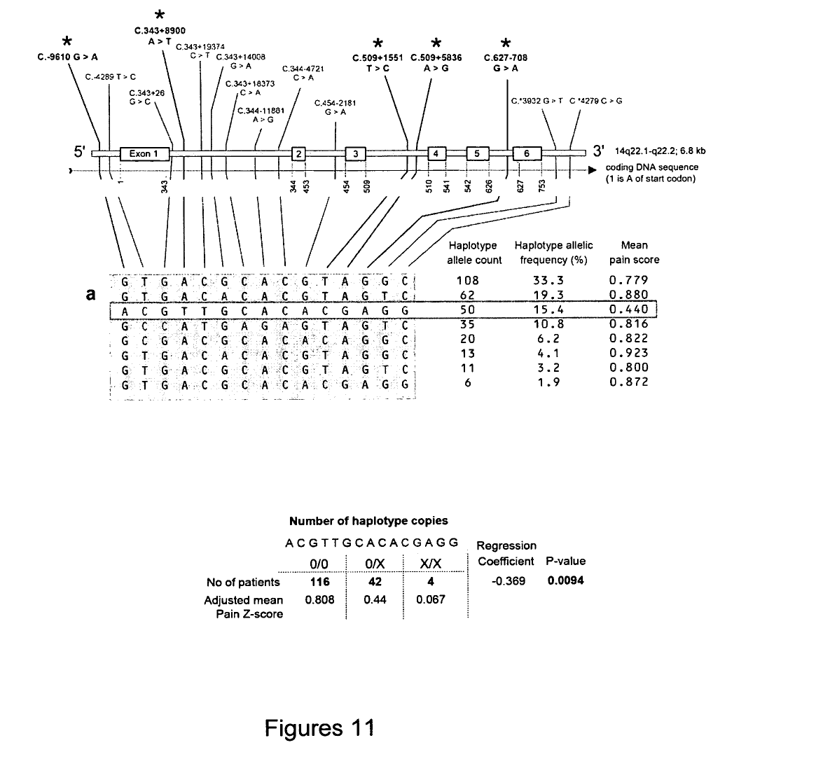

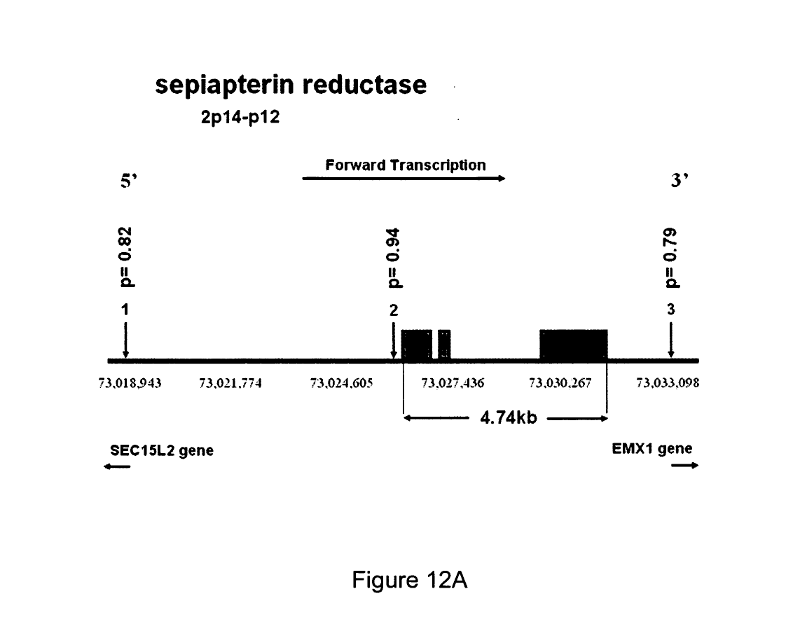

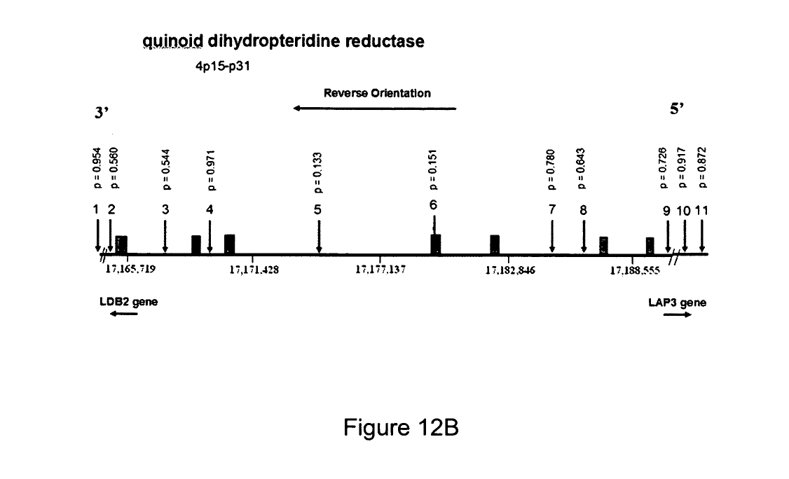

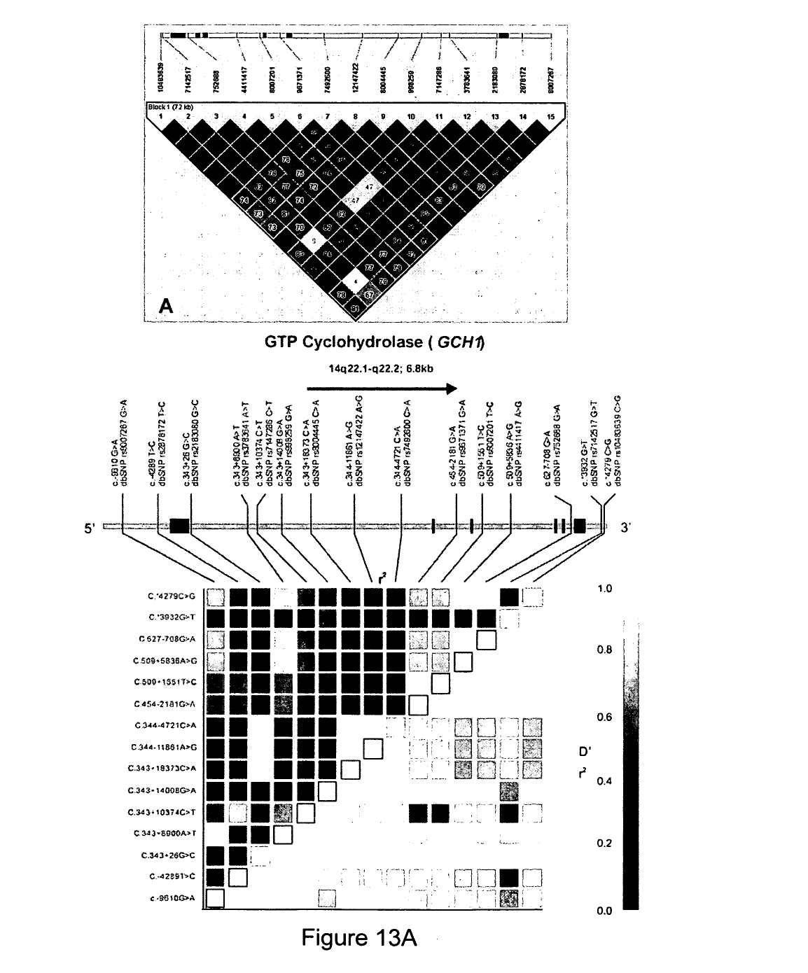

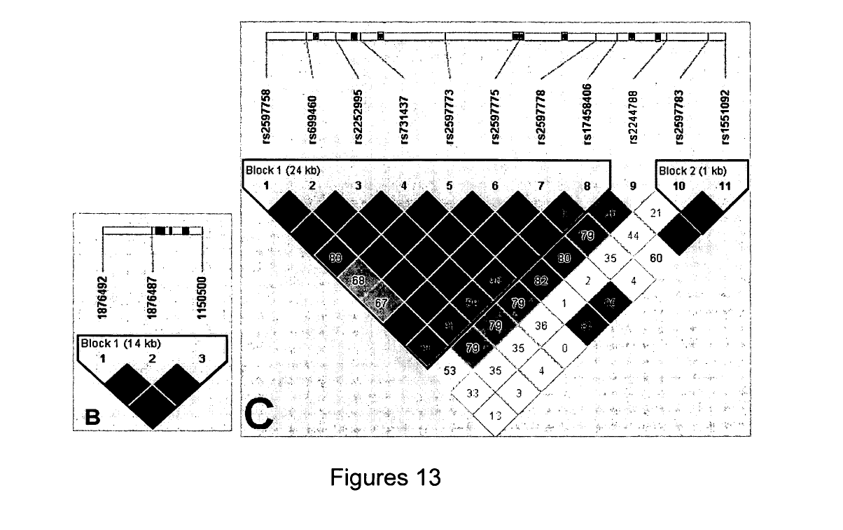

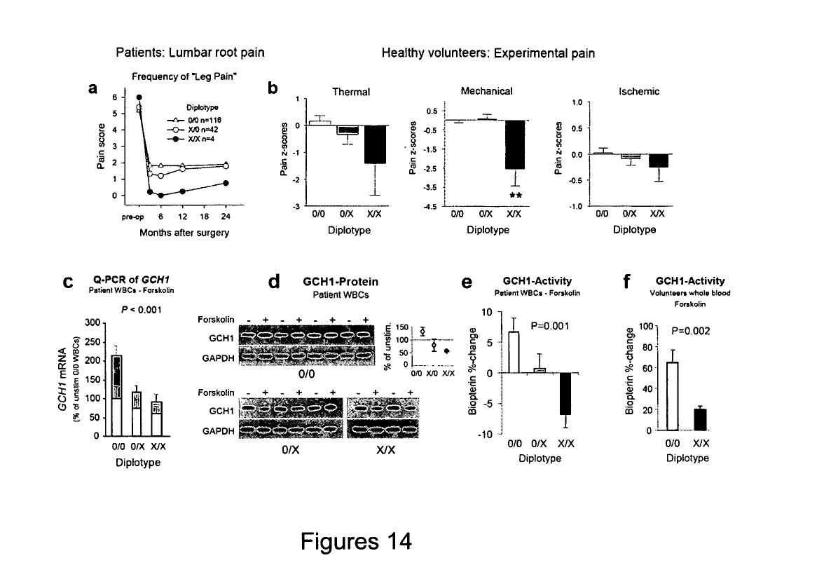

We next determined whether polymorphisms in the genes that code for GTP cyclohydrolase (GCH1), sepiapterin reductase (SPR), or dihydropteridine reductase (QDPR) are linked to a distinct pain phenotype in human patients. DNA from 168 Caucasian adults, participants in a prospective observational study of surgical discectomy for persistent lumbar root pain caused by intervertebral disc herniation, was collected (Atlas et al., Spine 21:1777-1786 (1996); Chang et al., J Am Geriatr Soc 53:785-792 (2005)). Prior to the analyses, a single primary endpoint, persistent leg pain over the first postoperative year, was specified as a reflection of neuropathic pain. Secondary endpoints were changes in levels of anxiety and depression over the first year postoperatively, adjusted for the magnitude of pain relief provided by the surgery. From these participants, 15 single nucleotide polymorphisms (SNPs), spaced evenly through GCH1 (FIGS. 11A and 13A; Table 3A), 3 SNPs in SPR (FIGS. 12A and 13B; Table 3B) and 11 SNPs in QDPR (FIGS. 12B and 13C, Table 3C), were genotyped using the 5′ exonuclease method (Shi et al., Biologicals 27:241-52 (1999)). Five SNPs in GCH1 (FIG. 11A) were significantly associated with low scores of persistent leg pain over the first postoperative year, pre-specified as the primary outcome. GCH1 and SPR each have a single conserved haplotype block 72 kb and 14 kb in size (FIGS. 13A and 13B), respectively, spanning the genes, while QDPR has at least 2 haploblocks (FIG. 13C). Five SNPs in GCH1 (FIG. 11A), but none in SPR or QDPR (FIGS. 12A and 12B; FIGS. 13B and 13C), were significantly associated with low scores of leg pain. GCH1 haplotypes could be determined in 162/168 patients. The haplotype analysis (FIG. 11A) identified one GCH1 haplotype with a population frequency of 15.4% that was highly associated with low scores of persistent leg pain (p=0.009). FIG. 14A shows representative raw pain scores over time for the frequency of leg pain at rest, one of four variables used to calculate the pain z-score. In 147 patients who completed the one-year questionnaire, the numbers of patients who reported that their leg pain was worse, unchanged, or only a little better one year after surgery were 0/4 (0%) of those with two copies of the protective haplotype, 4/41 (10%) of those with one copy, and 22/102 (22%) of those with no copies of this haplotype (FIG. 11B). Comparison of the haplotypes shows that two of the SNPs significantly associated with low pain scores (C.-9610G>A and C.343+8900A>T) are unique to the pain protective haplotype (FIG. 11A). These data indicate that GTP cyclohydrolase haplotype is a predictor of pain chronicity in humans; identification of GTP cyclohydrolase haplotype in a patient may therefore be used to determine if the patient has an altered susceptibility for developing chronic pain.

TABLE 3A
Locations and allelic frequencies of fifteen GCH1 markers

| | | | Allelic | | | | | | | |
|---|---|---|---|---|---|---|---|---|---|---|
| | Location | Allelic | frequency of | Number of | | | mean pain z-score | | | Regression |
| | relative to | variation | uncommon | patients* | | | for “Leg pain” | | | analysis |
| dbSNP ID | coding region | common > uncommon | allele [%] | 0/0 | 0/1 | 1/1 | 0/0 | 0/1 | 1/1 | p-value |
| rs8007267 | C.−9610 | G > A | 17.50 | 108 | 48 | 4 | 0.81 | 0.48 | 0.06 | 0.0128 |
| rs2878172 | C.−4289 | T > C | 37.42 | 64 | 71 | 24 | 0.92 | 0.57 | 0.69 | 0.1262 |
| rs2183080 | C.343 + 26 | G > C | 11.18 | 129 | 28 | 4 | 0.77 | 0.63 | 1.57 | 0.6424 |
| rs3783641 | C.343 + 8900 | A > T | 17.41 | 108 | 45 | 5 | 0.82 | 0.51 | 0.15 | 0.0212 |
| rs7147286 | C.343 + 10374 | C > T | 29.69 | 81 | 63 | 16 | 0.89 | 0.49 | 0.82 | 0.1256 |
| rs998259 | C.343 + 14008 | G > A | 25.63 | 89 | 60 | 11 | 0.67 | 0.79 | 0.95 | 0.2746 |
| rs8004445 | C.343 + 18373 | C > A | 10.94 | 129 | 27 | 4 | 0.78 | 0.63 | 1.58 | 0.6559 |
| rs12147422 | C.344 − 11861 | A > G | 11.25 | 128 | 28 | 4 | 0.76 | 0.66 | 1.56 | 0.5322 |
| rs7492600 | C.344 − 4721 | C > A | 11.25 | 128 | 28 | 4 | 0.76 | 0.67 | 1.57 | 0.5250 |
| rs9671371 | C.454 − 2181 | G > A | 25.63 | 87 | 61 | 10 | 0.81 | 0.59 | 0.32 | 0.0537 |
| rs8007201 | C.509 + 1551 | T > C | 25.63 | 90 | 58 | 12 | 0.81 | 0.61 | 0.21 | 0.0300 |
| rs4411417 | C.509 + 5836 | A > G | 18.13 | 109 | 44 | 7 | 0.81 | 0.54 | 0.18 | 0.0279 |
| rs752688 | C.627 − 708 | G > A | 18.01 | 110 | 44 | 7 | 0.80 | 0.54 | 0.18 | 0.0289 |
| rs7142517 | C.*3932 | G > T | 35.76 | 67 | 69 | 22 | 0.60 | 0.76 | 0.93 | 0.1360 |
| rs10483639 | C.*4279 | C > G | 18.13 | 109 | 44 | 7 | 0.79 | 0.58 | 0.19 | 0.0516 |

*0 = common allele, 1 = uncommon allele.

TABLE 3B
Locations and allelic frequencies of three SPR markers

| | SNP ID | SNP ID* | | Position* | | Allelic frequency (for |
|---|---|---|---|---|---|---|
| # | (CDS) | (NCBI) | Variation | (NCBI) | Location | allele 2) |
| 1 | HCV11938698 | rs1876492 | G > C | 73018943 | 5′ Intergenic | 0.92 |
| 2 | HCV11938855 | rs1876487 | C > A | 73026007 | 5′ Intergenic | 0.31 |
| 3 | HCV8882615 | rs1150500 | G > A | 73033098 | 3′ Intergenic | 0.08 |

TABLE 3C
Locations and allelic frequencies of eleven QDPR markers

| | | | | | | Allelic |
|---|---|---|---|---|---|---|
| | SNP ID | SNP ID* | | Position* | | frequency |
| # | (CDS) | (NCBI) | Variation | (NCBI) | Location | (for allele 2) |
| 1 | HCV15898885 | rs2597758 | A > G | 17161750 | Intergenic/Unknown | 0.647 |
| 2 | HCV8939566 | rs699460 | G > T | 17164555 | UTR 3′ | 0.686 |
| 3 | HCV3000237 | rs2252995 | G > T | 17166609 | Intron | 0.689 |
| 4 | HCV3000236 | rs17957134 | G > A | 17168414 | Intron | 0.344 |
| 5 | HCV15898932 | rs2597773 | T > G | 17174436 | Intron | 0.323 |
| 6 | HCV25474129 | rs2597775 | G > A | 17179651 | Silent Mutation | 0.322 |
| 7 | HCV1321013 | rs2597778 | A > G | 17185023 | Intron | 0.686 |
| 8 | HCV3000231 | rs17458406 | A > G | 17186505 | Intron | 0.853 |
| 9 | HCV15898956 | rs2244788 | G > C | 17189993 | UTR 5′ | 0.665 |
| 10 | HCV1321003 | rs2597783 | A > G | 17192960 | Intergenic/Unknown | 0.691 |
| 11 | HCV1321000 | rs1551092 | G > A | 17194130 | Intergenic/Unknown | 0.422 |

NCBI IDs and SNP physical locations are from the National Center for Biotechnology Information database, August 2005 or the Ensemble Database v.38, April 2006. In few patients not all SNPs could be determined.

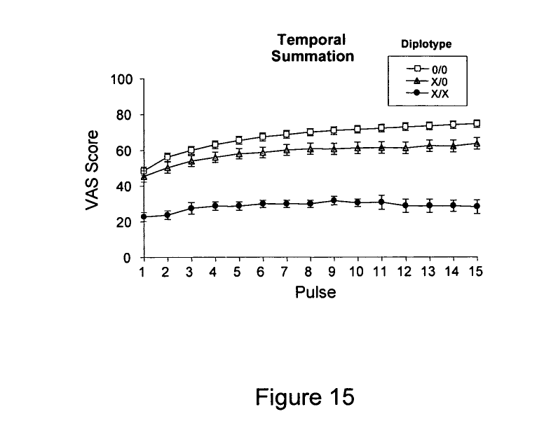

We next explored whether this “pain protective haplotype” is also associated with reduced heat, ischemic, and pressure pain sensitivity in two independent cohorts of healthy volunteers (see Methods described below and Table 4). Individuals carrying two copies of the “pain protective haplotype” are significantly less sensitive to mechanical pain and tend to be less sensitive to heat pain and ischemic pain (FIG. 14B). In one cohort, individuals with this diplotype (n=4) showed significantly reduced temporal summation of heat pain (FIG. 15). This finding was not replicated in the second cohort. Heterozygotes for the haplotype also tend to be less pain sensitive and tend to show reduced temporal summation to heat pain as compared to those without a copy of this haplotype (FIGS. 14B and 15). These data indicate that GTP cyclohydrolase is additionally a regulator of acute pain sensitivity in humans.

Table 4, shown below, shows the associations of heat, mechanical, and ischemic pain with the number of copies of the “pain protective haplotype” in two independent cohorts of healthy volunteers. One cohort was examined at the University of North Carolina at Chapel Hill (UNC) and the second cohort was examined at the University of Florida (UF). Each individual pain measure was standardized to unit normal deviates (z-scores) with a mean of zero and standard deviation of one. Subjects who did not carry the “pain protective haplotype” X were grouped as 0/0, subjects carrying one X haplotype were grouped as X/0, and subjects carrying two copies of X haplotype were grouped as X/X. Independent association study analyses for each cohort and the combined cohorts are presented.

TABLE 4

| | | | | Mechanical | | Ischemic | |
|---|---|---|---|---|---|---|---|
| Cohort | Diplotype | Thermal z-score | SEM | z-score | SEM | z-score | SEM |
| UF | OO (n = 240) | −0.09 | 0.11 | −0.13 | 0.11 | −0.02 | 0.11 |
| | XO (n = 89) | 0.19 | 0.2 | 0.27 | 0.2 | −0.05 | 0.17 |
| | XX (n = 6) | −1.13 | 0.28 | −1.47 | 1.1 | −0.7 | 0.29 |
| | P value | 0.14 | | 0.028 | | 0.57 | |
| UNC | OO (n = 144) | 0.42 | 0.43 | 0.20 | 0.30 | 0.06 | 0.14 |
| | XO (n = 64) | −0.85 | 0.65 | −0.20 | 0.45 | −0.17 | 0.22 |
| | XX (n = 4) | −1.32 | 2.58 | −4.16 | 1.79 | 0.36 | 0.87 |
| | P value | 0.23 | | 0.0508 | | 0.62 | |
| Combined | OO (n = 384) | 0.15 | 0.22 | −0.004 | 0.13 | 0.02 | 0.09 |
| | XO (n = 153) | −0.33 | 0.37 | 0.07 | 0.24 | −0.09 | 0.13 |
| | XX (n = 10) | −1.41 | 1.18 | −2.54 | 0.89 | −0.25 | 0.28 |
| | P value | 0.25 | | 0.006 | | 0.58 | |

Leukocyte Studies

GCH1 mRNA and protein expression and BH4 synthesis were analyzed in EBV-immortalized leukocytes of patients who participated in the lumbar root pain study (Atlas et al., Spine 21:1777-1786 (1996); Chang et al., J Am Geriatr Soc 53:785-792 (2005)). Baseline expression (mRNA and protein) of GCH1 and BH4 levels did not significantly differ between carriers and non-carriers of the haplotype. Since GCH1 transcription increases in response to cAMP, acting through regulatory elements located in the proximal promoter (Hirayama et al., J Neurochem 79:576-587 (2001); Kapatos et al., J Biol Chem 275:5947-5957 (2000)), the cells were stimulated with forskolin (10 μM, 12 h) to increase adenyl cyclase activity. Forskolin increased GCH1 mRNA (FIG. 14C), protein (FIG. 14D) and BH4 production (FIG. 14E) in patients with no copies of the pain protective haplotype. The upregulation by forskolin of the GCH1 transcript was significantly reduced in leukocytes with one or two copies of the pain protective haplotype (FIG. 14C). In contrast to non-carriers, GCH1 protein levels in WBCs (FIG. 14D) and biopterin concentrations in WBC culture supernatants (FIG. 14E) fell below baseline in homozygous haplotype carriers suggesting that the haplotype may modify protein stability. Cells of heterozygous carriers had an intermediate phenotype (FIGS. 14D and 14E). We further analyzed biopterin in whole blood of healthy homozygous 0/0 and X/X volunteers. Baseline biopterin levels were slightly higher in homozygous carriers of the haplotype compared with non-carriers. Following forskolin treatment (10 μM, 24 h), biopterin increased by about 60% in non-carriers, as compared with 20% in homozygous carriers of the haplotype (FIG. 14F). Differences between WBCs and whole blood (falling levels versus reduced increase) may be caused by BH4 recycling via QDPR in erythrocytes.

We also found that LPS, like forskolin, induced GCH1 to a lesser extent in cells from individuals with the pain protective haplotype as compared to individuals without the pain protective haplotype. Previous work has shown that stimulation with LPS, IL-1, TNF, and interferon gamma, like cAMP, increases cellular GTPCH levels and activity. Accordingly, we believe that cells from individuals carrying the pain protective haplotype or having reduced pain sensitivity will exhibit reduced levels/activity of GCH1 when contacted with an inflammatory cytokine or an interferon.

Tetrahydrobiopterin synthesis increases in rat sensory neurons in response both to axonal injury and peripheral inflammation. Blocking the increased BH4 synthesis by independently inhibiting two successive enzymes in the synthesis cascade reduces neuropathic and inflammatory pain and in contrast, BH4 administration produces pain in naïve animals and enhances inflammatory and neuropathic pain sensitivity. Furthermore, a haplotype of GCH1 that reduces its upregulation in response to a forskolin challenge is protective against persistent neuropathic pain and associated with reduced sensitivity to experimental pain in humans. We therefore have identified both a novel pathway involved in the production and modulation of pain and a genetic marker of pain sensitivity.

Materials and Methods for GTP Cyclohydrolase Studies

The following materials and methods were used to generate the results presented in Example 1.

Microarray Hybridization, Real Time RT-PCR, Slot Blot

Extraction of RNA, hybridization on the Affymetrix RGU34A chip in triplicate, and analysis of the array data were as described (Costigan et al., BMC Neurosci 3:16 (2002)). For Northern slot blots total RNA was transferred to nylon membranes, hybridized with ³²P-labeled cDNA probes, and quantified using cyclophilin for normalization. Quantitative real-time PCR was performed using the Sybr green detection system with primer sets designed on Primer Express. Specific PCR product amplification was confirmed with gel electrophoresis. Transcript regulation was determined using the relative standard curve method per manufacturer's instructions (Applied Biosystems).

In Situ Hybridization

Fresh frozen DRGs were cut at 18 μm, postfixed, and acetylated. Riboprobes were obtained by in vitro transcription of cDNA and labeled with digoxigenin (Dig-labeling kit, Roche). Sections were hybridized with 200 ng/ml of sense or antisense probes in a prehybridization mix (Blackshaw and Snyder, J Neurosci 17:8083-8092 (1997)) and incubated with anti-Dig-AP (1:1000), developed with NBT/BCIP/levamisole, embedded in glycerol/gelatin or subjected to post in situ immunostaining. Primary antibodies: sheep Dig-AP 1:1000 (Roche), mouse NF200 1:4000 (Sigma), rabbit ATF-3 1:300 (SantaCruz). FITC-labeled Griffonia simplicifolia isolectin B4 (Sigma) 1:500. Blocking and antibody incubations in 1% blocking reagent (Roche).

Nerve Injury Models

Adult male Sprague Dawley rats (150-200 g, Charles River Laboratories) were used. For the SNI model two branches of the sciatic nerve, the common peroneal and the tibial nerve, were ligated and sectioned distally. For the CCI model the sciatic nerve was constricted with three Dexon 4/0 ligatures. For the SNL model, the L5 spinal nerve was tightly ligated. All surgical procedures were under isoflurane anesthesia. For the Formalin test 50 μl of 5% formaldehyde solution were injected into a hindpaw and flinches were counted per minute up to 60 min. Paw inflammation was induced with 50 μl complete Freund's adjuvant (CFA) injected into a hindpaw. Nociceptive analysis was done blinded, and animals were fully habituated to the room and test cages. Mechanical allodynia was assessed with graded strength monofilament von Frey hairs (0.0174-20.9 gram, log scaled), cold allodynia with the acetone test and heat hyperalgesia with the Hargreaves test. Drugs (Sigma) were injected intraperitoneally or intrathecally through a spinal catheter, osmotic pumps were used for infusion. Control animals received vehicle. L4/5 DRG and spinal cord tissue was processed for QRT-PCR, Western blotting, in situ hybridization and immunofluorescence studies.

Inflammatory Models

For the Formalin test 50 μl of 5% formaldehyde solution were injected into one hindpaw and flinches were counted per minute up to 60 min. Two hours after formalin injection animals were perfused with 4% PFA in 1×PBS, the spinal cord was dissected and subjected to cFos immunostaining (rabbit pAb Santa Cruz 1:500). For paw inflammation 50 μl complete Freund's Adjuvant (CFA) was injected into the paw.

Nociceptive Behavior

Animals were fully habituated and experiments performed blinded. Threshold for eliciting a withdrawal reflex to graded strength monofilament von Frey hairs (0.0174-20.9 g) was measured to assess mechanical allodynia. To measure cold allodynia, a drop of acetone was applied to the plantar hindpaw, and the time the animal spent licking, shaking or lifting the paw was measured (Tegeder et al., J Neurosci 24:1637-1645 (2004)). Paw withdrawal latency to radiant heat (lamp with 8 V, 50 W) assessed heat evoked pain (Ugo Basile).

Drug Treatment

DAHP was dissolved in 1:1 polyethylene glycol (PEG400) and 1×PBS, pH 7.4 (15 mg/ml) and administered i.p. or intrathecally (250 μg/kg/h; 5 μl/h). For all i.t. injections/infusions a spinal catheter (Recathco) was used and implanted as described (Kunz et al., Pain 110:409-418 (2004)). Infusions with an osmotic pump (Alzet). 6R-BH4 in ACSF was injected i.t. (10 μg, single 10 μl injection). N-acetyl-serotonin in 1×PBS pH 7.4 containing 3% ethanol was delivered by i.t. infusion (100 μg/kg/h; 5 μl/h). Control animals received the appropriate vehicle. All drugs from Sigma-Aldrich.

Plasma and CSF Concentrations of DAHP

Concentrations of DAHP were determined LC/MS-MS on a tandem quadrupole mass spectrometer (PE Sciex API 3000; Applied Biosystems). Extraction was by acetonitrile precipitation; chromatographic separation was performed on a Nucleosil C18 Nautilus column (125×4 mm I.D., 5 μm particle size, 100 Å pore size). Mobile phase was acetonitrile:water (80:20%, v/v), and formic acid (0.1%, v/v). Flow rate was 0.2 ml/min, and injection volume was 5 μl. DAHP eluted at 4.7 min. Mass spectrometer in positive ion mode, 5200 V, 400° C., auxiliary gas flow 6 l/min. The mass transition for the MRM was m/z 127→60. Quantification with Analyst software V1.1 (Applied Biosystems). Coefficient of variation over the calibration range of 10-4000 ng/ml <5%.

Immortalization of Leukocytes and Forskolin Stimulation

Peripheral blood lymphocytes were immortalized with EBV transfection. WBCs were stimulated with PHA in RPMI media, EBV was then added and cells were incubated at 37° C., 4.5% CO₂, 90% relative humidity. Immortalized cells were stimulated with 10 μM forskolin for 12 h.

Tissue Concentrations of Neopterin and Biopterin

Homogenized tissue was oxidized with iodine, and pteridines were extracted on Oasis MCX cartridges. Concentrations of total biopterin, neopterin, and the internal standard rhamnopterin were determined by LC/MS-MS. LC analysis under gradient conditions on a Nucleosil C8 column; MS-MS analyses on an API 4000 Q TRAP triple quadrupole mass spectrometer. Precursor-to-product ion transitions of m/z 236→192 for biopterin, m/z 252→192 for neopterin, m/z 265→192 for rhamnopterin were used for the MRM. Linearity from 0.1-50 ng/ml. The coefficient of correlation for all measured sequences was at least 0.99. The intra-day and inter-day variability was <10%.

Electrophysiology

Miniature EPSCs were recorded at −70 mV by whole cell patch clamp in adult rat transverse spinal cord slices (Baba et al., Mol Cell Neurosci 24:818-830 (2003)). Intracellular [Ca]_{I} was measured fluorometrically (ΔF 340/380) in cultured adult DRG neurons loaded with fura-2. 6R-BH4 (0.3-10 μM), DEA-NONOate (50 μM), and L-NAME (10-100 μM) were applied using a multibarrel fast drug delivery system.

Data Analysis

Data are means ±SEM. The number of animals per group was 9-12. Areas under the “effect versus time” curves (AUC) were calculated using the linear trapezoidal rule and compared with Student's t-test or univariate analysis of variance (ANOVA) with subsequent t-tests employing a Bonferroni alpha-correction for multiple comparisons. All other data were analyzed with univariate ANOVA or ANOVA for repeated measurements. P at 0.05 for all tests.

Human Genetic Studies

We genotyped 15 single nucleotide polymorphisms (SNPs), spaced evenly through GCH1, using the 5′ exonuclease method (Primer sets and probes in Table 6A). GCH1 haplotypes were identified in-silico using PHASE software, which implements a modified Expectation/Maximization (EM) algorithm to reconstruct haplotypes from population genotype data. Linkage disequilibrium (D′) between SNPs was used to describe the non-independence of alleles (FIG. 13A).

Chronic Lumbar Root Pain: Pain Outcome

We collected DNA from 168 Caucasian adults who participated in a prospective observational study of surgical diskectomy for persistent lumbar root pain (demographic data in Table 5 below). Between 1990 and 1992, approximately half of the active spine surgeons in Maine enrolled patients requiring diskectomy for lumbar root pain in a prospective observational study (Atlas et al., Spine 21:1777-1786 (1996)). Patients completed questionnaires pre-operatively, and at 3, 6, and 12 months postoperatively, and then annually through year 10. Pain outcome: leg pain was assessed by four items: Frequencies in the past week of “leg pain”, and of “leg pain after walking”, were rated as “never (0 points),” “very rarely (1),” “a few times (2),” “about ½ the time (3),” “usually (4),” “almost always (5),” and “always (6).” “Percent improvement in pain frequency” scores were calculated by subtracting frequency scores from the baseline score and dividing by the baseline score. Improvements in “leg pain” or in “leg pain after walking” since surgery were rated as “pain completely gone (6),” “much better (5),” “better (4),” “a little better (3),” “about the same (2),” “a little worse (1),” and “much worse (O).” For each variable in each patient, we calculated an area-under-the-curve score for the first year, and converted this score to a z-score by comparing the patient to the rest of the cohort. The z-score expresses the divergence of the experimental result x from the most probable result p as a number of standard deviations, calculated as z=(x−μ)/σ. The primary pain outcome variable was the mean of these four z-scores. Genotype-phenotype associations for each polymorphism were sought using the equation: leg pain over first year=a+b (number of copies of uncommon allele: 0, 1, or 2)+c (sex)+d (age)+e (workman's compensation status)+f (delay in surgery after initial enrollment)+g (Short-Form 36 (SF-36) general health scale)+error.

TABLE 5
Demographic data of the Lumbar Root Pain study

| | | Number of copies of the pain | | |
|---|---|---|---|---|
| | | protective haplotype | | |
| | All patients | 0 | 1 | 2 |
| Number of patients | 162 | 116 | 42 | 4 |
| Mean age (range) | 40 (20-78) | 42 (20-78) | 44 (26-67) | 35 (31-40) |
| Males/Females | 102/60 | 76/40 | 25/17 | 1/3 |
| Length of pain episode | 110/52 | 82/34 | 25/17 | 3/1 |
| before surgery | | | | |
| ≦6 months/ | | | | |
| >6 months | | | | |

Experimental Pain Sensitivity in Healthy Subjects

In two separate cohorts of healthy volunteers we analyzed the association of heat, ischemic and mechanical pain with GCH1 diplotypes. One cohort was examined at the University of North Carolina at Chapel Hill (UNC) and the second cohort was examined at the University of Florida (UF). For the association studies, 384 subjects who did not carry the “pain protective haplotype” X as defined by the lumbar root pain study were grouped as 0/0, 153 subjects carrying one X haplotype were grouped as X/0, and 10 subjects carrying two copies of the X haplotype were grouped as X/X.

UNC Cohort: This sample group consisted of 212 healthy women aged 18 to 34 years of age (mean age 22.8). Experimental procedures used to assess pain perception are described in (Diatchenko et al., Hum Mol Genet 14:135-143 (2005)). Briefly, measures of heat pain threshold and tolerance (° C.) were averaged across three anatomical test sites, i.e. arm, cheek and foot. Pressure pain thresholds (kg) were assessed over the temporalis and masseter muscles, the temporomandibular joint and the ventral surface of the wrists. Temporal summation of heat pain was assessed by applying fifteen 53° C. heat pulses to the thenar region of the right hand. Subjects were instructed to rate their perception of each pulse using a verbal numerical analog scale using values between “0” and “19” to rate the intensity of non-painful warmth, and “20” (pain threshold) to “100” (most intense pain imaginable) to rate the intensity of heat pain. Ischemic pain threshold and tolerance (seconds) were assessed with the submaximal effort tourniquet procedure.

UF Cohort: This sample group consisted of 192 healthy female and 143 healthy male volunteers aged 18 to 52 years of age (mean age 24.0). Experimental procedures are described in Hastie et al. (Pain 116:227-237 (2005)). Briefly, heat pain threshold and tolerance (° C.) were assessed on the volar forearm, and 0 to 100 ratings of repetitive suprathreshold heat pain were assessed at 2 temperatures, 49 and 52° C. Pressure pain threshold (kg) was assessed at three sites, the masseter and trapezius muscle, and dorsal forearm over the ulna. Ischemic pain threshold and tolerance (seconds) were assessed via the submaximal effort tourniquet procedure.

In order to combine the data across the two cohorts, each subject's value for a given pain measure was standardized to unit normal deviates (z-scores) with a mean of zero and standard deviation of one. Differences between the diplotype groups were determined using one way ANOVA. For the UNC cohort, the effect of the diplotype on the differences in curve profiles (FIG. 15) were analyzed using a one-way ANOVA followed by a Bonferroni adjustment for post-hoc testing (p<0.001 for each diplotype comparison).

Genotyping Methods

SNP markers: The physical position and frequency of minor alleles (>0.05) from a commercial database (Celera Discovery System, CDS, July, 2005) were used to select SNPs. 5′ nuclease assays could be designed for fifteen GCH1, three SPR, and eleven QDPR SNPs and genotyped in a highly accurate fashion. These panels of approximately equally-spaced markers covered each gene region plus 4-6 kb upstream and 4-6 kb downstream of each gene. Allele frequencies of all markers and their locations in their respective genes are shown in Tables 3A-3C.

Genomic DNA: Genomic DNA was extracted from lymphoblastoid cell lines and diluted to a concentration of 5 ng/μl. Two-μl aliquots were dried in 384-well plates.

Polymerase chain reaction (PCR) amplification: Genotyping was performed by the 5′ nuclease method using fluorogenic allele-specific probes. Oligonucleotide primer and probes sets were designed based on gene sequences from the CDS, July 2005. Primers and detection probes for each locus in each gene are listed in Tables 6A-6C below.

TABLE 6A

Primer and probe sequences for 5′ nuclease genotyping of
fifteen GCH1 markers

| | | | |
|---|---|---|---|
| # | dbSNP# | Primers and probes | Sequences |

| | | | |
|---|---|---|---|
| 1 | rs8007267 | Assay on demand #1545138 | |
| | | (ABI, Ca) | |
| 2 | rs2878172 | Forward primer | GAGGCAGGGACAGAGTTCAG |
| | | | (SEQ ID NO:1) |
| 2 | | Reverse primer | AGAAGAACAGGCAGATGCTAAGAG |
| | | | (SEQ ID NO:2) |
| 2 | | Allele 1 probe (FAM) | TGAGGTGCACTCTCTATTA |
| | | | (SEQ ID NO:3) |
| 2 | | Allele 2 probe (VIC) | TGAGGTGCATTTCTATTAG |
| | | | (SEQ ID NO:4) |
| 3 | rs2183080 | Forward primer | CCGCGGGCTGCTAGAG |
| | | | (SEQ ID NO:5) |
| 3 | | Reverse primer | GGCAACTCCGGAAACTTCCT |
| | | | (SEQ ID NO:6) |
| 3 | | Allele 1 probe (FAM) | GGTGCTTGGAGGAAA |
| | | | (SEQ ID NO:7) |
| 3 | | Allele 2 probe (VIC) | GGTGCTTGCAGGAA |
| | | | (SEQ ID NO:8) |
| 4 | rs3783641 | Forward primer | TCCATGCCTGGGCATTCC |
| | | | (SEQ ID NO:9) |
| 4 | | Reverse primer | CCAAATACTAGACTCAAATTACAGTCCTCAT |
| | | | (SEQ ID NO:10) |
| 4 | | Allele 1 probe (FAM) | TCATTTGCCAGTGATTT |
| | | | (SEQ ID NO:11) |
| 4 | | Allele 2 probe (VIC) | CTCATTTGCCTGTGATTT |
| | | | (SEQ ID NO:12) |
| 5 | rs7147286 | Forward primer | ACAGCTTCTCTTTGGCATAACTGAA |
| | | | (SEQ ID NO:13) |
| 5 | | Reverse primer | TCAGTTTTGCAGTGTTTGTTTTCAAGT |
| | | | (SEQ ID NO:14) |
| 5 | | Allele 1 probe (FAM) | CCAACGTCACTACTCTTG |
| | | | (SEQ ID NO:15) |
| 5 | | Allele 2 probe (VIC) | CCAATGTCACTACTCTTG |
| | | | (SEQ ID NO:16) |
| 6 | rs998259 | Assay on demand #7593515 | |
| | | (ABI, Ca) | |
| 7 | rs8004445 | Assay on demand #9866676 | |
| | | (ABI, Ca) | |
| 8 | rs12147422 | Forward primer | GTGGTGTTGTTGTAGACAAACCTTT |
| | | | (SEQ ID NO:17) |
| 8 | | Reverse primer | GCATTCTGTTTCCTACGGTTGGT |
| | | | (SEQ ID NO:18) |
| 8 | | Allele 1 probe (FAM) | GCTTTCGTTTTGTTTGT |
| | | | (SEQ ID NO:19) |
| 8 | | Allele 2 probe (VIC) | GCTTTCATTTTGTTTGTG |
| | | | (SEQ ID NO:20) |
| 9 | rs7492600 | Forward primer | TGTTTGAAGTTAGCTTTATTAAGGTGTCACT |
| | | | (SEQ ID NO:21) |
| 9 | | Reverse primer | GGGTGGCTATATAACTGCATACGTT |
| | | | (SEQ ID NO:22) |
| 9 | | Allele 1 probe (FAM) | AAATTTACCTACTTTACA |
| | | | (SEQ ID NO:23) |
| 9 | | Allele 2 probe (VIC) | AAATTTAACTACTTTACATG |
| | | | (SEQ ID NO:24) |
| 10 | rs9671371 | Forward primer | AAGGAATCTTTGAAAGGGAATCTATTGGT |
| | | | (SEQ ID NO:25) |
| 10 | | Reverse primer | CCAAGCCACTAACTCTCTCTATCCT |
| | | | (SEQ ID NO:26) |
| 10 | | Allele 1 probe (FAM) | CAAATTAGGCACAGAAA |
| | | | (SEQ ID NO:27) |
| 10 | | Allele 2 probe (VIC) | AGCAAATTAGACACAGAAA |
| | | | (SEQ ID NO:28) |
| 11 | rs8007201 | Forward pnmer | GGTGGTCCTGATATTTCTCAATTCTGT |
| | | | (SEQ ID NO:29) |
| 11 | | Reverse primer | CAGGAACAACTTTAGAGGGCAGTT |
| | | | (SEQ ID NO:30) |
| 11 | | Allele 1 probe (FAM) | CTACCCCAGCAATC |
| | | | (SEQ ID NO:31) |
| 11 | | Allele 2 probe (VIC) | AAAACTACTCCAGCAATC |
| | | | (SEQ ID NO:32) |
| 12 | rs4411417 | Assay on demand #11164699 | |
| | | (ABI, Ca) | |
| 13 | rs752688 | Assay on demand #9866644 | |
| | | (ABI, Ca) | |
| 14 | rs7142517 | Forward primer | ACGCAGTGTGTCTTCCTTCAC |
| | | | (SEQ ID NO:33) |
| 14 | | Reverse primer | TCGACCTCATCAATTACATTTTCATGACA |
| | | | (SEQ ID NO:34) |
| 14 | | Allele 1 probe (FAM) | CTTTGTCGGACAGAGC |
| | | | (SEQ ID NO:35) |
| 14 | | Allele 2 probe (VIC) | CTTTGTCGGCCAGAGC |
| | | | (SEQ ID NO:36) |
| 15 | rs10483639 | Forward primer | GGAAAAGGAGGAAGAATAAAAAATGCATTCTAA |
| | | | (SEQ ID NO:37) |
| 15 | | Reverse primer | AAATGCCTGGGTGTGTGTATGTA |
| | | | (SEQ ID NO:38) |
| 15 | | Allele 1 probe (FAM) | CCTGAGACGAAGTTG |
| | | | (SEQ ID NO:39) |
| 15 | | Allele 2 probe (VIC) | CCTGAGAGGAAGTTG |
| | | | (SEQ ID NO:40) |

TABLE 6B

Primer and probe sequences for 5′ nuclease
genotyping of three SPR markers

| | | |
|---|---|---|
| # | Primers and probes | Sequences |

| | | |
|---|---|---|
| 1 | Forward primer | GCTGACACTGGCATCTTCTAATCGT |
| | | (SEQ ID NO:41) |
| | Reverse primer | TGTCCCTGCTTACAGTAGTCTCT |
| | | (SEQ ID NO:42) |
| | Allele 1 probe (FAM) | AGTGACCGCCCCC |
| | | (SEQ ID NO:43) |
| | Allele 2 probe (VIC) | CAGTGACCCCCCCC |
| | | (SEQ ID NO:44) |
| 2 | Assay on demand | |
| | #11938855 | |
| | (ABI, Ca) | |
| 3 | Assay on demand | |
| | #8882615 | |
| | (ABI, Ca) | |

TABLE 6C

Primer and probe sequences for 5′ nuclease
genotyping of eleven QDPR markers

| | | |
|---|---|---|
| # | Primers and probes | Sequences |

| | | |
|---|---|---|
| 1 | Forward primer | GAGAGCTGGTAGTCTTCATTCCATT |
| | | (SEQ ID NO:45) |
| | Reverse primer CTAGAATCATGGACTGCTTGGAAGT | |
| | | (SEQ ID NO:46) |
| | Allele 1 probe (FAM) | CTACTCATCCGTTGGTG |
| | | (SEQ ID NO:47) |
| | Allele 2 probe (VIC) | CCTACTCATCCATTGGTG |
| | | (SEQ ID NO:48) |
| 2 | Assay on demand #8939566 | |
| | (ABI, Ca) | |
| 3 | Assay on demand #3000237 | |
| | (ABI, Ca) | |
| 4 | Forward primer | GCTACTCTGAGATTCCGTCTGATG |
| | | (SEQ ID NO:49) |
| | Reverse primer GGTGGTCTTGGGAGGTCTCT | |
| | | (SEQ ID NO:50) |
| | Allele 1 probe (FAM) | CTGAGGATGCGTTGCA |
| | | (SEQ ID NO:51) |
| | Allele 2 probe (VIC) | CTGAGGATGCATTGCA |
| | | (SEQ ID NO:52) |
| 5 | Assay on demand #15898932 | |
| | (ABI, Ca) | |
| 6 | Forward primer | CCAGGGCAGCCTTTGC |
| | | (SEQ ID NO:53) |
| | Reverse primer | CTACCAAGCATCTCAAGGAAGGA |
| | | (SEQ ID NO:54) |
| | Allele 1 probe (FAM) | CTCCTGACCTTGGCTG |
| | | (SEQ ID NO:55) |
| | Allele 2 probe (VIC) | CCTCCTAACCTTGGCTG |
| | | (SEQ ID NO:56) |
| 7 | Forward primer | GCTTATTTGTATTTTCTATATCATACATGCATCACTTCT |
| | | (SEQ ID NO:57) |
| | Reverse primer | CGTGGGTCTGCTTTTCATTTAGTTG |
| | | (SEQ ID NO:58) |
| | Allele 1 probe (FAM) | ACTTTCCTTGGTAATCT |
| | | (SEQ ID NO:59) |
| | Allele 2 probe (VIC) | CACTTTCCTTAGTAATCT |
| | | (SEQ ID NO:60) |
| 8 | Forward primer | AAATGGAATATCACACATCTACAAAGAGGTT |
| | | (SEQ ID NO:61) |
| | Reverse primer | TTTAGGTAATTTTGTATTTTATAGTTTATGGTAAGCTTTGTTTT |
| | | (SEQ ID NO:62) |
| | Allele 1 probe (FAM) | AATAATTCTCCAGGTTACTG |
| | | (SEQ ID NO:63) |
| | Allele 2 probe (VIC) | AAATAATTCTCCAGATTACTG |
| | | (SEQ ID NO:64) |
| 9 | Forward primer | TCCCGCAGCTCCGAATG |
| | | (SEQ ID NO:65) |
| | Reverse primer | CGCGCGTTCCCTCTTG |
| | | (SEQ ID NO:66) |
| | Allele 1 probe (FAM) | CCTCGAGCCCGAGCG |
| | | (SEQ ID NO:67) |
| | Allele 2 probe (VIC) | CCTCGAGCCGGAGCG |
| | | (SEQ ID NO:68) |
| 10 | Forward primer | CCGCTACATAGTCAGGTGAAGATTG |
| | | (SEQ ID NO:69) |
| | Reverse primer | TCCATGCTTCCTACAACCACATC |
| | | (SEQ ID NO:70) |
| | Allele 1 probe (FAM) | CAGAAGCCTCTGCAGAGA |
| | | (SEQ ID NO:71) |
| | Allele 2 probe (VIC) | CAGAAGCCTCTACAGAGA |
| | | (SEQ ID NO:72) |
| 11 | Assay on demand #1321003 | |
| | (ABI, Ca) | |

Reactions were performed in a 5 μl volume containing 2.25 μl TE (Assays On Demand) or 2.375 μl TE (Assays By Design), 2.5 μl PCR Master Mix (ABI, Foster City, Calif.), 10 ng genomic DNA, 900 nM of each forward and reverse primer, and 100 nM of each reporter and quencher probe. DNA was incubated at 50° C. for 2 min and at 95° C. for 10 min, and amplified on an ABI 9700 device for 40 cycles at 92° C. (Assays on Demand) or 95° C. (Assays By Design) for 15 s and 60° C. for 1 min. Allele-specific signals were distinguished by measuring endpoint 6-FAM or VIC fluorescence intensities at 508 nm and 560 nm, respectively, and genotypes were generated using Sequence Detection V. 1.7 (ABI).

Genotyping error rate was directly determined by re-genotyping 25% of the samples, randomly chosen, for each locus. The overall error rate was <0.005. Genotype completion rate was 0.99.

Inference of haplotypes: Haplotype phases—i.e., how the directly measured SNP alleles were distributed into two chromosomes in each patient—were inferred by the expectation-maximization (EM) algorithm (SAS/Genetics, Cary, N.C., USA).

### EXAMPLE 2

### KCNS1 Pain Protective Haplotypes

KCNS1 Involvement in Chronic Pain

Voltage-gated potassium channels form the largest and most diversified class of ion channels and are present in both excitable and nonexcitable cells. Such channels generally regulate the resting membrane potential and control the shape and frequency of action potentials. The potassium voltage-gated channel, delayed-rectifier, subfamily S, member 1 (KCNS1) or voltage-gated potassium channel 9.1 (KV9.1) gene encodes a potassium channel alpha subunit expressed in a variety of neurons, including those of the inferior colliculus. The protein encoded by KCNS1 is not functional alone; it can form heteromultimers with member 1 and with member 2 (and possibly other members) of the Shab-related subfamily of potassium voltage-gated channel proteins. This gene belongs to the S subfamily of the potassium channel family. KCNS1 is very highly expressed in the brain but is not detectable in other tissues. Within the brain, highest expression levels were found in the main olfactory bulb, cerebral cortex, hippocampal formation, habenula, basolateral amygdaloid nuclei, and cerebellum.

The opening of some K(+) channels plays an important role in the antinociception induced by agonists of many G-protein-coupled receptors (e.g., alpha(2)-adrenoceptors, opioid, GABA(B), muscarinic M(2), adenosine A(1), serotonin 5-HT(1A) and cannabinoid receptors). Several specific types of K(+) channels are involved in antinociception. The most widely studied are the ATP-sensitive K(+) channels. Drugs that open K(+) channels by direct activation (such as openers of neuronal K(v)₇ and K(ATP) channels) produce antinociception in models of acute and chronic pain, suggesting that other neuronal K(+) channels (e.g., K(v) 1.4 channels) may represent an interesting target for the development of new K(+) channel openers with antinociceptive effects (Salinas et al., J. Biol. Chem. 272:24371-24379 (1997); Bourinet et al., Curr. Top. Med. Chem. 5:539-46. (2005); Ocana et al., Eur. J. Pharmacol. 500:203-19 (2004)). A reduction in K(+) channels after nerve injury may increase the risk of developing ectopic or spontaneous firing of neurons. Decreased K(+) channel opening may also reduce efficacy of opiate or other analgesic treatment.

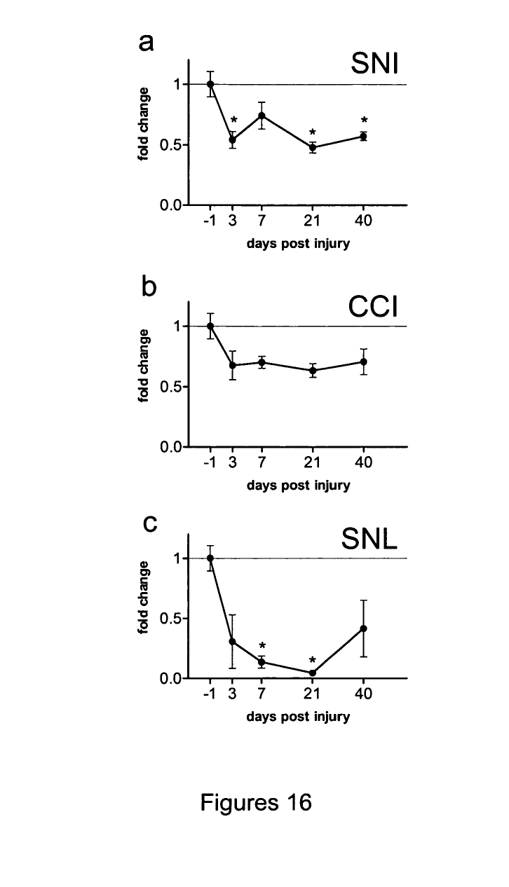

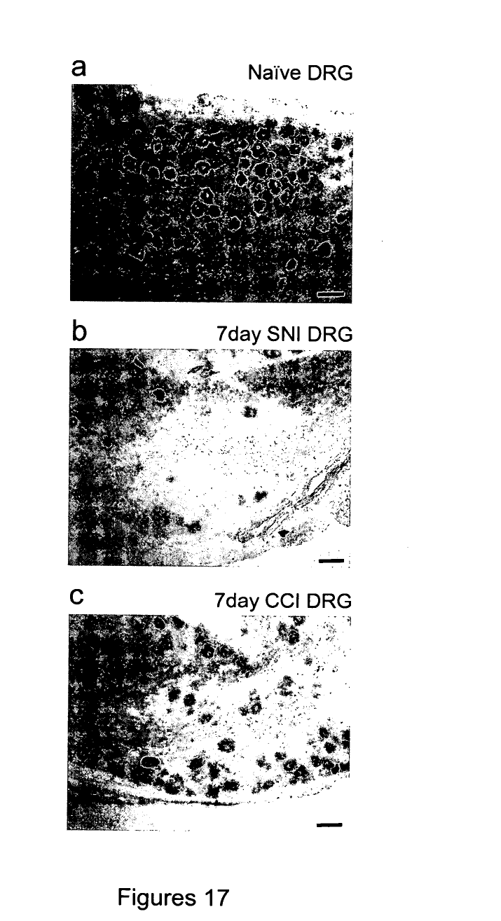

In a manner similar to the identification of the genes involved in BH4 synthesis, the KCNS1 gene has been identified as being involved in chronic pain. Downregulation of the KCNS1 transcript in all three models of peripheral neuropathic pain (FIGS. 16A-16C) over time (3 to 40 days) in the rat DRG using microarrays was observed. These results were validated by in situ hybridization of KCNS1 mRNA (FIGS. 17A-17C).

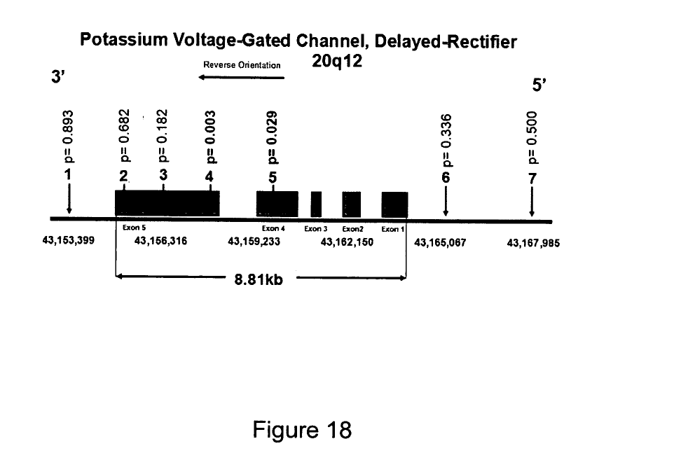

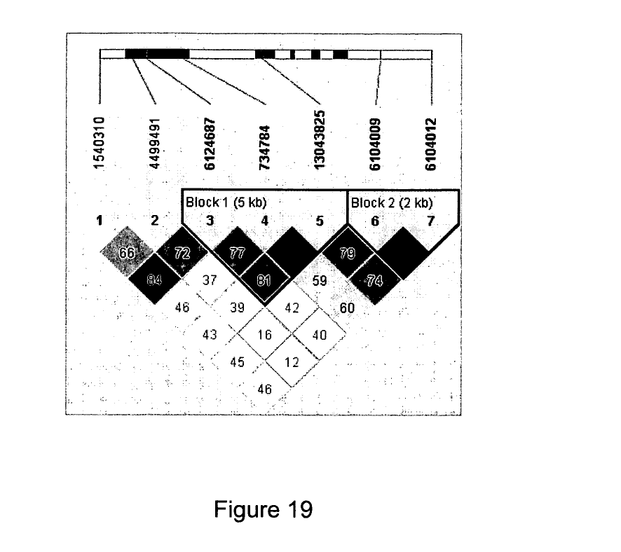

KCNS1 is located on chromosome 20q12. Previously, no KCNS1 mutations or sequence variants had been used for association studies. Because of the lack of available putative functional KCNS1 variants, comprehensive haplotype-based analyses were performed in our chronic pain association study using a series of loci chosen for haplotype informativeness including known synonymous and non-synonymous mutations in the coding region (see markers numbers 4 and 5 respectively; FIG. 18, Table 7). We, for the first time, identified KCNS1 haplotype structure and investigated associations with pain scores in our population, using a panel of evenly spaced single nucleotide polymorphism (SNP) markers with sufficient density. A total of seven markers were genotyped using the 5′ exonuclease method (Shi et al., Biologicals 27:241-52 (1999)). KCNS1 had at least two haplotype blocks, with almost perfect linkage disequilibrium (LD) between markers 4 and 5 (FIG. 19). Single SNP analysis revealed that those two SNPs were significantly associated with low scores of sciatica pain (Table 8). From haplotype and diplotype analysis, a common haplotype (frequency >0.53), ‘111 or GTG’, was identified from a reconstruction of markers 3, 4, and 5 in Block 1, as being highly associated with low scores of chronic leg pain, particularly in subjects with two copies of this “low pain” protective haplotype (p<0.004, Table 8). Allele 1 in SNP #4 (rs 734784) is adenine, representing codon ATT, which encodes Ile. A switch to nucleotide G at the same position changes this codon to GTT, which encodes Val. This variant is most strongly associated with greater pain. This change, the change in SNP #5, or another unidentified variant associated with the haplotype may therefore influence KCNS1 function.

TABLE 7

| | | | Celera | NCBI | P |
|---|---|---|---|---|---|
| SNP | dB SNP ID | Polymorphism | hCV | Location | value |
| 1 | rs1540310 | Intergenic | 7591825 | 43,153,399 | 0.893 |
| 2 | rs4499491 | UTR 3′ | 2457091 | 43,154,833 | 0.682 |
| 3 | rs6124687 | UTR 3′ | 2457088 | 43,155,431 | 0.182 |
| 4 | rs734784 | Ile 489 Val | 2457087 | 43,157,041 | 0.003 |
| 5 | rs13043825 | Glu 86 Glu | 2457085 | 43,160,569 | 0.029 |
| 6 | rs6104009 | Intergenic | 2457073 | 43,165,788 | 0.336 |
| 7 | rs6104012 | Intergenic | 26338135 | 43,167,985 | 0.5 |

TABLE 8

| Location | SNP name | |
|---|---|---|
| 40428628 | KCNS1_0 | |
| 40430062 | KCNS1_1434 | |
| 40430660 | KCNS1_2032 | used |
| 40432270 | KCNS1_3642 | used |
| 40435798 | KCNS1_7170 | used |
| 40441017 | KCNS1_12389 | |
| 40443214 | KCNS1_14586 | |

Haplotype frequencies and means

| Effect | Dependent | Haplotype | LSMean | COUNT | PERCENT |
|---|---|---|---|---|---|
| haplotype | grand_z_1y | 111 | 0.6331 | 86 | 53.29 |
| haplotype | grand_z_1y | 121 | 0.912804 | 32 | 19.67 |
| haplotype | grand_z_1y | 122 | 0.888743 | 14 | 8.84 |
| haplotype | grand_z_1y | 211 | 0.197293 | 3 | 1.69 |
| haplotype | grand_z_1y | 222 | 0.988307 | 26 | 16.10 |
| | | | | | 99.59 |

Diplotype analysis

| Effect | Dependent | diplotype_n | No. of patients | % | LSMean | ProbtDiff |
|---|---|---|---|---|---|---|
| Diplotype_n | grand_z_1y | 111/others | 36 | 22 | 0.67408 | |
| Diplotype_n | grand_z_1y | Others/others | 125 | 78 | 1.17527 | 0.00404 |

In Kv9.1, the SNP that changed isoleucine to valine was significant at 0.003 in the Maine low back pain post surgical patients. The primer and probe sequences used in this study for the 5′ nuclease genotyping of the seven KCNS1 markers are shown in Table 9.

TABLE 9

| | | |
|---|---|---|
| # | Primers and probes | Sequences |

| | | |
|---|---|---|
| 1 | Forward primer | AGAGAGAGGCATATGACTCAAGTGA |
| | | (SEQ ID NO:73) |
| | Reverse primer | GTATCATCCTGCTCACAGTTCCAA |
| | | (SEQ ID NO:74) |
| | Allele 1 probe (FAM) | CCCAGGAGAGAGTC |
| | | (SEQ ID NO:75) |
| | Allele 2 probe (VIC) | TCCCAGGACAGAGTC |
| | | (SEQ ID NO:76) |
| 2 | Forward primer | GCCATTCTCTCTGCTTGGAGTA |
| | | (SEQ ID NO:77) |
| | Reverse primer | CCTGAGCAAGTGACAATCTAACCT |
| | | (SEQ ID NO:78) |
| | Allele 1 probe (FAM) | CCCCCCTGGAACC |
| | | (SEQ ID NQ:79) |
| | Allele 2 probe (VIC) | CTCCCCACTGGAACC |
| | | (SEQ ID NO:80) |
| 3 | Forward primer | GACCTCCTTTTCAGTCTTGTTCACA |
| | | (SEQ ID NO:81) |
| | Reverse primer | CTGGGTGCCAAGCTCAGA |
| | | (SEQ ID NO:82) |
| | Allele 1 probe (FAM) | TTTTTGAGGGCCAGGTC |
| | | (SEQ ID NO:83) |
| | Allele 2 probe (VIC) | CCTTTTTGAGGTCCAGGTC |
| | | (SEQ ID NO:84) |
| 4 | Assay on demand | |
| | #2457087 | |
| | (ABI, Ca) | |
| 5 | Forward primer | GCCGCCTCGTCGTAGTC |
| | | (SEQ ID NO:85) |
| | Reverse primer | TGGGCCGCCTGCA |
| | | (SEQ ID NO:86) |
| | Allele 1 probe (FAM) | CGGAGGAGCAGGC |
| | | (SEQ ID NO:87) |
| | Allele 2 probe (VIC) | CGGAGGAACAGGC |
| | | (SEQ ID NO:88) |
| 6 | Assay on demand | |
| | #2457073 | |
| | (ABI, Ca) | |
| 7 | Forward primer | CTCCTGGCCTCCCATAGC |
| | | (SEQ ID NO:89) |
| | Reverse primer | CCTAGCTAGAGAGTTGCATGACAT |
| | | (SEQ ID NO:90) |
| | Allele 1 probe (FAM) | CCCAGGCCTCTCT |
| | | (SEQ ID NO:91) |
| | Allele 2 probe (VIC) | CTCCCAGACCTCTCT |
| | | (SEQ ID NO:92) |

### EXAMPLE 3

Methods and Kits for Diagnosing a Propensity toward Pain Sensitivity, Developing Acute or Chronic Pain, or a Propensity to Develop a BH4-related Disorder

The present invention provides methods and kits useful in the diagnosis of pain sensitivity, the diagnosis of a propensity for, or risk of developing, acute or chronic pain in a subject, based on the discovery of allelic variants and haplotypes in the GCH1 and KCNS1 genes, or the risk of developing a BH4-related disorder based on the discovery of allelic variants and haplotypes in the GCH1 gene. Additional methods and kits are based the discovery that the GCH1 haplotype associated with reduced pain sensitive results in a reduced GCH1 expression and activity in leukocytes when challenged with forskolin, an agent which increases cellular cyclic AMP levels.

The results generated from use of such methods and kits can be used, for example, to determine the dosing or choice of an analgesic administered to the subject, whether to include the subject in a clinical trial involving an analgesic, whether to carry out a surgical procedure on the subject or to choose a method for anesthesia, whether to administer a neurotoxic treatment to the subject, or the likelihood of pain development in the subject (e.g., as part of an insurance risk analysis or choice of job assignment).

In addition, results generate from performing these methods can be used in conjunction with clinical trial data. The gold standard for proof of efficacy of a medical treatment is a statistically significant result in a clinical trial. By incorporating the presence or absence of a pain-protective haplotype into analysis of clinical trial data, it can be possible to generate statistically significant differences between the experimental arm and control groups of the trial. In particular, we believe GCH1 and KCNS1 genotypes or haplotypes can explain some of the variance observed within clinical trials. In particular, the genotypes or haplotypes described herein can be included in statistical analysis of pain trials, or other clinical trials for which GCH1 may be relevant, such as studies of vascular disease or mood.

These methods and kits are described in greater detail below.

Methods and Kits for Identifying Allelic Variants in a Subject

The methods for identifying an allelic variant in a subject can include the identification of the presence or absence of a polymorphism associated with an altered pain phenotype as well as a determination of the number of polymorphic alleles (e.g., 0, 1, or 2 alleles). Kits of the invention can include primers (e.g., 2, 3, 4, 8, 10, or more primers) which can be used to amplify genomic or mRNA to determine the presence or absence of an allelic variant. While the presence of a single allelic variant can be used for this analysis, the presence of multiple pain-protective alleles (for example, multiple pain-protective SNPs) is preferred for diagnostic purposes. Preferably, at least 4, more preferably, at least 8, 10 or 12, and most preferably at least 15 pain-protective allelic variants (e.g., SNPs) are detected and used for diagnostic or predictive purposes. Moreover, while the presence of a single copy of a pain protective allelic variant or haplotype indicates a reduced propensity for pain sensitivity or development of acute or chronic pain, the presence of two copies is further indicative of decreased pain sensitivity or acute or chronic pain propensity.

Detection of allelic variants can be performed by any method for nucleic acid analysis. For example, diagnosis can be accomplished by sequencing a portion of the genomic locus of the GCH1 or KCNS1 gene known to contain a polymorphism (e.g., a SNP) associated with an altered propensity to develop pain sensitivity or acute or chronic pain from a sample taken from a subject. This sequence analysis, as is known in the art and described herein, indicates the presence or absence of the polymorphism, which in turn elucidates the pain sensitivity and pain response profile of the subject.

In addition to sequencing, allelic variant and haplotype analysis may also be achieved, for example, using any PCR-based genotyping methods known in the art. Any primer capable of amplifying regions of the GCH1 or KCNS1 genes known to contain pain-protective polymorphisms may be utilized. Primers particularly useful for GCH1 and KCNS1 genotyping are listed in Tables 6A and 9, respectively, and allelic variants that correlate with altered pain risk are shown in Tables 1 and 2 and FIG. 11A. In an exemplary diagnostic assay, a biological sample may be obtained from a patient and subjected to PCR (e.g., using primers in Table 6A or 8) to amplify a region (e.g., a region shown in Table 3A or Table 8) that contains a pain-protective polymorphism. For a polymorphism that occurs in an intronic region, analysis of genomic DNA is generally used. If a polymorphism occurs in a transcribed region of a gene (e.g., in the coding sequence or promoter region), analysis of mRNA may instead be utilized. The presence or absence of the polymorphism indicates whether the subject is at altered risk for enhanced pain sensitivity or the development of acute or chronic pain.

Other methods of genotyping that may be used in the invention include the TaqMan 5′ exonuclease method, which is fast and sensitive, as well as hybridization to microsphere arrays and fluorescent detection by flow cytometry. Chemical assays, including allele specific hybridization (ASH), single base chain extension (SBCE), allele specific primer extension (ASPE), and oligonucleotide ligation assay (OLA), can be implemented in conjunction with microsphere arrays. Fluorescence classification techniques allow genotyping of up to 50 diallelic markers simultaneously in a single well. Typically, it requires less than one hour to analyze a 96-well plate permitting analysis of tens of thousands of genotypes per day.

Additional methods of genotype analysis that can be used in the invention include the SNPlex genotyping system, which is based on oligonucleotide ligation/PCR assay (OLA/PCR) technology and the ZipChute Mobility Modifier probes for multiplexed SNP genotyping. This method allows for the performance of over 200,000 genotypes per day with high accuracy and reproducibility. In one particular example, this method allows for identification of 48 SNPs simultaneously in a single biological sample with the ability to detect 4,500 SNPs in parallel in 15 minutes. While all of the above represent exemplary genotyping methods, any method known in the art for nucleic acid analysis may be used in the invention.

Methods and Kits for Identifying Altered GCH1 Expression or Activity in a Cell

The invention features methods that can be used to determine whether a subject has an altered sensitivity to pain or an altered risk of developing acute or chronic pain or developing an BH4-related disorder. In particular, the invention features methods and kits for determining if GCH1 expression or activity is altered (e.g., increased or decreased) in cells such as leukocytes following a challenge such as administration of an agent that increases cellular cyclic AMP (cAMP) levels, administration of LPS, administration of an inflammatory cytokine (e.g., IL-1, TNF), or administration of an interferon (e.g., interferon gamma). Any agent that increases cAMP levels may be used in the methods of the invention. For example, agents such as adenyl cyclase activators (e.g., forskolin), dexamethasone, cholera toxin, cAMP analogs (e.g., 8-bromo-cyclic AMP, 8-(4-chlorophenylthio)cyclic AMP, N⁶, O^{2′}-dibutyryl cylic AMP), cyclic AMP phosphodiesterase inhibitors (e.g., 3-isobutyl-1-methylxanthine, flavinoids described by Beretz et al., Cell Mol Life Sci 34:1054-1055, 1978, or any phosphodiesterase inhibitor known in the art), thyrotropin, thyrotripin releasing hormone, vasoactive intestinal polypeptide, and ethanol can be used to increase cAMP levels in a cell.

GCH1 expression or activity may assayed, for example, by measuring levels of GCH1 mRNA (e.g., using a microarray, QT-PCR, northern blot analysis, or any other method known in the art) or GCH1 protein (e.g., using an antibody based detection method such as a Western blot or ELISA). GCH1 activity can be measured using an intermediate or product of the BH4 pathway such as neopterin, biopterin, or BH4. In general, expression or activity of GCH1 in a cell treated with an agent that increases cAMP levels (e.g., forskolin) is measured and then compared to a baseline value or baseline values. A change in GCH1 expression or activity relative to the baseline value(s) is therefore indicative of the test subject's pain sensitivity, the test subject's risk of developing acute or chronic pain, or the test subject's risk of developing an BH4-related disorder.

A baseline value for use in the diagnostic methods of the invention may be established by several different means. In one example, a positive control is used as the baseline value. Here, GCH1 expression or activity level from an individual with the GCH1 pain-protective haplotype treated with an agent is measured and used as a baseline value. Thus, an increase (e.g., of at least 3%, 5%, 10%, 20%, 30%, 40%, 50%, 75%, 90%, 100%, or 200%) in GCH1 expression or activity in the test subject as compared to the baseline value is indicative of increased pain sensitivity or an increased risk of developing acute or chronic pain or developing an BH4-related disorder as compared to an individual with the GCH1 pain protective haplotype.

A baseline value may also be established by averaging GCH1 expression or activity values over a number of individuals. For example, the GCH1 expression or activity in cells from individuals (e.g., at least 2, 5, 10, 20, 50, 100, 200, or 500 individuals) with the GCH1 pain protective haplotype may be used to establish a baseline value for a positive control. A negative control value may likewise be established from a group of individuals (e.g., at least 2, 5, 10, 20, 50, 100, 200, or 500 individuals), for example, either (a) from individuals selected at random or (b) from individuals known to lack copies of the GCH1 pain protective haplotype.

A sample from a test subject may also be compared to multiple baseline values, e.g., established from two or three groups of individuals. For example, three groups of individuals (e.g., where each group independently consists of at least 2, 5, 10, 20, 50, 100, or 200 individuals) may be used to establish three baseline values. In this approach, subjects are separated into the three groups based on whether they have zero, one, or two copies of the GCH1 pain protective haplotype. The level of GCH1 expression or activity upon treatment of cells from each individual with a composition that increases cAMP levels is measured. The average value of GCH1 expression or activity for each group can thus be calculated from these measurements, thereby establishing three baseline values. The value measured from treated sample of the test subject is then compared to the three baseline values. The test subject's pain sensitivity, risk of developing acute or chronic pain, or risk of developing an BH4-related disorder can accordingly be determined on this basis of this comparison.

### OTHER EMBODIMENTS

All patents, patent applications, and publications mentioned in this specification are herein incorporated by reference to the same extent as if each independent patent, patent application, or publication was specifically and individually indicated to be incorporated by reference.

What is claimed is:

1. A method for predicting pain sensitivity, diagnosing the risk of developing acute or chronic pain, or diagnosing the risk of developing a BH4-associated disorder in a mammalian subject, said method comprising determining the presence or absence of an allelic variant in a GTP cyclohydrolase (GCH1) nucleic acid in a biological sample from said subject, said allelic variant correlating with pain sensitivity, development of acute or chronic pain, or development of a BH4-associated disorder.

2. The method of claim 1, wherein said GCH1 allelic variant is present in a haplotype block located within human chromosome 14q22.1-14q22.2.

3. The method of claim 2, wherein said GCH1 allelic variant comprises a SNP selected from the group consisting of rs6572984, rs17128017, rs10151500, rs10136966, rs841, rs987, rs17253577, rs11624963, rs752688, rs7493025, rs2004633, rs7493033, rs17253584, rs10139369, rs10150825, rs11848732, rs17253591, rs10143089, rs13329045, rs10131232, rs10133662, rs10133941, rs13329058, rs9672037, rs7161034, rs7140523, rs11626298, rs17128021, rs10129528, rs4411417, rs2878168, rs11461307, rs7153186, rs7153566, rs7155099, rs11444305, rs11439363, rs7155309, rs1952437, rs8007201, rs11412107, rs12587434, rs17128028, rs12589758, rs2878169, rs28532361, rs12879111, rs0129468, rs11620796, rs2149483, rs7147200, rs4462519, rs9671371, rs9671850, rs9671455, rs28481447, rs12884925, rs8010282, rs8010689, rs8011751, rs7156475, rs17128033, rs28643468, rs2183084, rs10137881, rs2878170, rs12323905, rs10138301, rs12323579, rs10138429, rs12323582, rs7141433, rs7141483, rs7141319, rs2183083, rs2183082, rs2183081, rs7492600, rs8009470, rs10144581, rs12323758, rs10145097, rs13368101, rs10134163, rs13367062, rs4402455, rs7493427, rs10311834, rs9743836, rs4363780, rs7493265, rs10312723, rs4363781, rs7493266, rs10312724, rs11627767, rs11850691, rs11627828, rs11626155, rs2878171, rs10220344, rs10782424, rs3965763, rs0146709, rs10146658, rs10147430, rs17128050, rs12147422, rs28477407, rs10143025, rs10133449, rs10133650, rs3945570, rs28757745, rs28542181, rs7155501, rs3825610, rs3783637, rs3783638, rs3783639, rs3825611, rs11158026, rs11158027, rs10873086, rs11626210, rs8004445, rs8004018, rs8010461, rs9805909, rs8009759, rs10444720, rs4901549, rs3783640, rs10136545, rs10139282, rs8020798, rs10498471, rs28417208, rs11845055, rs10498472, rs998259, rs8101712, rs11312854, rs11410453, rs10782425, rs10149080, rs17128052, rs8003903, rs10645822, rs10132356, rs13366912, rs12885400, rs7147286, rs7147040, rs7147201, rs17832263, rs10133661, rs3783641, rs3783642, rs12432756, rs10134429, rs10598935, rs10545051, rs17128057, rs8016730, rs8017210, rs11844799, rs12883072, rs10131633, rs10131563, rs10149945, rs8019791, rs8019824, rs8018688, rs10138594, rs10141456, rs9972204, rs2149482, rs28413055, rs2183080, rs28458175, and rs1753589.

4. The method of claim 1, wherein said allelic variant is present in the promoter or in a regulatory region of the GCH1 gene.

5. The method of claim 1, wherein said GCH1 allelic variant comprises an A at position C.-9610 or a T at position C.343+8900, or comprises an A at position C.-9610 and a T at position C.343+8900.

6. The method of claim 5, wherein said GCH1 allelic variant comprises an A at position C.-9610, C at position C.-4289, G at position C.343+26, T at position C.343+8900, T at position C.343+10374, G at position C.343+14008, C at position C.343+18373, A at position C.344-11861, C at position C.344-4721, A at position C.454-2181, C at position C.509+1551, G at position C.509+5836, A at position C.627-708, G at position C.*3932, and G at position C.*4279 of the GCH1 sequence.

7. The method of claim 1, wherein said BH4-related disorder is a cardiovascular disease or a neurological disease.

8. The method of claim 7, wherein said cardiovascular disease is atherosclerosis, ischemic reperfusion injury, cardiac hypertrophy, hypertension, vasculitis, myocardial infarction, or cardiomyopathy.

9. The method of claim 7, wherein said neurological disease is depression, a neurodegenerative disorder, a movement disorder, or an autonomic disturbance.

10. The method of claim 1, wherein said method comprises determining whether said nucleic acid sample comprises one copy or multiple copies of said allelic variant.

11. The method of claim 1, wherein said acute pain is one or more of mechanical pain, heat pain, cold pain, ischemic pain, or chemical-induced pain.

12. The method of claim 1, wherein said pain is peripheral or central neuropathic pain, inflammatory pain, migraine-related pain, headache-related pain, irritable bowel syndrome-related pain, fibromyalgia-related pain, arthritic pain, skeletal pain, joint pain, gastrointestinal pain, muscle pain, angina pain, facial pain, pelvic pain, claudication, postoperative pain, post traumatic pain, tension-type headache, obstetric pain, gynecological pain, or chemotherapy-induced pain.

13. The method of claim 1, wherein said mammal is a human.

14. The method of claim 1, wherein the presence or absence of said allelic variant is determined by nucleic acid sequencing or is determined by PCR analysis.

15. The method of claim 1, wherein said method is used to determine the dosing or choice of an analgesic administered to said subject.

16. The method of claim 1, wherein said method is used to determine whether to include said subject in a clinical trial involving an analgesic.

17. The method of claim 1, wherein said method is used to determine whether to carry out a surgical procedure on said subject, to determine whether to administer a neurotoxic treatment to said subject, or to choose a method for anesthesia.

18. The method of claim 17, wherein said surgical procedure involves nerve damage or treatment of nerve damage.

19. The method of claim 1, wherein said method is used to determine the likelihood of pain development in said subject as part of an insurance risk analysis or choice of job assignment.

20. A method for predicting pain sensitivity or diagnosing the risk of developing acute or chronic pain in a mammalian subject, said method comprising determining the presence or absence of an allelic variant in a potassium voltage-gated channel, delayed-rectifier, subfamily S, member 1 (KCNS1) nucleic acid in a biological sample from said subject, said allelic variant correlating with pain sensitivity or development of acute or chronic pain.

21. The method of claim 20, wherein said allelic variant comprises a SNP selected from the group consisting of rs6124683, rs4499491, rs8118000, rs6124684, rs6124685, rs12480253, rs6124686, rs6124687, rs6031988, rs6065785, rs1054136, rs17341034, rs6031989, rs7264544, rs734784, rs6104003, rs6104004, rs11699337, rs6017486, rs962550, rs7261171, rs6104005, rs13043825, rs7360359, rs8192648, rs6073642, rs6130749, rs6073643, rs6104006, rs6031990, rs8122867, rs8123330, and rs3213543.

22. The method of claim 20, wherein said allelic variant comprises an A at position 43,157,041 of the KCNS1 sequence.

23. The method of claim 22, wherein said KCNS1 allelic variant comprises a G at position 43,155,431, A at position 43,157,041, and C at position 43,160,569 of the KCNS1 sequence.

24. A method for predicting pain sensitivity, diagnosing the risk of developing acute or chronic pain, or diagnosing the risk of developing a BH4-associated disorder in a mammalian subject, said method comprising the steps of:
(a) contacting a biological sample comprising a cell from said subject with a composition that increases the level of cyclic AMP in said cell, comprises lipopolysaccharide (LPS), or comprises an inflammatory cytokine; and
(b) measuring the expression or activity of GTP cyclohydrolase (GCH1) in said sample, wherein said expression or activity, when compared to a baseline value, is indicative of whether said patient has altered pain sensitivity or is diagnostic of the risk of developing acute or chronic pain or developing a BH4-associated disorder in said subject.

25. The method of claim 24, wherein a decrease in GCH1 expression or activity is indicative of decreased pain sensitivity or decreased risk of developing acute or chronic pain.

26. The method of claim 24, wherein said measuring of GCH1 activity comprises measuring neopterin or biopterin levels in said cell.

27. The method of claim 24, wherein said cell is a leukocyte.

28. The method of claim 24, wherein said composition comprises a phosphodiesterase inhibitor or an adenyl cyclase activator.

29. The method of claim 28, wherein said adenyl cyclase activator is forskolin.

30. A kit for predicting pain sensitivity, diagnosing the risk of developing acute or chronic pain, diagnosing the risk of developing an BH4-related disorder in a mammalian subject, said kit comprising:
(a) a set of primers for amplification of a sequence comprising an allelic variant in a GCH1 gene; and
(b) instructions for use.

31. The kit of claim 30, wherein said GCH1 allelic variant is present in a haplotype block located within human chromosome 14q22.1-14q22.2.

32. The kit of claim 31, wherein said GCH1 allelic variant comprises a SNP selected from the group consisting of rs6572984, rs17128017, rs10151500, rs10136966, rs841, rs987, rs17253577, rs11624963, rs752688, rs7493025, rs2004633, rs7493033, rs17253584, rs10139369, rs10150825, rs11848732, rs17253591, rs10143089, rs13329045, rs10131232, rs10133662, rs10133941, rs13329058, rs9672037, rs7161034, rs7140523, rs11626298, rs17128021, rs10129528, rs4411417, rs2878168, rs11461307, rs7153186, rs7153566, rs7155099, rs11444305, rs11439363, rs7155309, rs1952437, rs8007201, rs11412107, rs12587434, rs17128028, rs12589758, rs2878169, rs28532361, rs12879111, rs0129468, rs11620796, rs2149483, rs7147200, rs4462519, rs9671371, rs9671850, rs9671455, rs28481447, rs12884925, rs8010282, rs8010689, rs8011751, rs7156475, rs17128033, rs28643468, rs2183084, rs10137881, rs2878170, rs12323905, rs10138301, rs12323579, rs10138429, rs12323582, rs7141433, rs7141483, rs7141319, rs2183083, rs2183082, rs2183081, rs7492600, rs8009470, rs10144581, rs12323758, rs10145097, rs13368101, rs10134163, rs13367062, rs4402455, rs7493427, rs10311834, rs9743836, rs4363780, rs7493265, rs10312723, rs4363781, rs7493266, rs10312724, rsl 1627767, rs11850691, rs11627828, rs11626155, rs2878171, rs10220344, rs10782424, rs3965763, rs10146709, rs10146658, rs10147430, rs17128050, rs12147422, rs28477407, rs10143025, rs10133449, rs10133650, rs3945570, rs28757745, rs28542181, rs7155501, rs3825610, rs3783637, rs3783638, rs3783639, rs3825611, rs11158026, rs11158027, rs10873086, rs11626210, rs8004445, rs8004018, rs8010461, rs9805909, rs8009759, rs10444720, rs4901549, rs3783640, rs10136545, rs10139282, rs8020798, rs10498-471, rs28417208, rs11845055, rs10498472, rs998259, rs8011712, rs11312854, rs11410453, rs10782425, rs10149080, rs17128052, rs8003903, rs10645822, rs10132356, rs13366912, rs12885400, rs7147286, rs7147040, rs7147201, rs17832263, rs10133661, rs3783641, rs3783642, rs12432756, rs10134429, rs10598935, rs10545051, rs17128057, rs8016730, rs8017210, rs11844799, rs12883072, rs10131633, rs10131563, rs10149945, rs8019791, rs8019824, rs8018688, rs10138594, rs10141456, rs9972204, rs2149482, rs28413055, rs2183080, rs28458175, and rs1753589.

33. The kit of claim 31, wherein said GCH1 allelic variant comprises an A at position C.-9610, C at position C.-4289, G at position C.343+26, T at position C.343+8900, T at position C.343+10374, G at position C.343+14008, C at position C.343+18373, A at position C.344-11861, C at position C.344-4721, A at position C.454-2181, C at position C.509+1551, G at position C.509+5836, A at position C.627-708, G at position C.*3932, and G at position C.*4279 of the GCH1 sequence.

34. The kit of claim 30, wherein said allelic variant is present in the promoter region or in a regulatory region of the GCH1 gene.

35. The kit of claim 30, wherein said BH4-related disorder is a cardiovascular disease or neurological disorder.

36. A kit for predicting pain sensitivity or diagnosing the risk of developing acute or chronic pain in a mammalian subject, said kit comprising:
(a) a set of primers for amplification of a sequence comprising an allelic variant in a KCNS1 gene; and
(b) instructions for use.

37. The kit of claim 36, wherein said KCNS1 allelic variant is present in a haplotype block located within human chromosome 20q12.

38. The kit of claim 36, wherein said allelic variant comprises a SNP selected from the group consisting of rs6124683, rs4499491, rs8118000, rs6124684, rs6124685, rs12480253, rs6124686, rs6124687, rs6031988, rs6065785, rs1054136, rs17341034, rs6031989, rs7264544, rs734784, rs6104003, rs6104004, rs11699337, rs6017486, rs962550, rs7261171, rs6104005, rs13043825, rs7360359, rs8192648, rs6073642, rs6130749, rs6073643, rs6104006, rs6031990, rs8122867, rs8123330, and rs3213543.

39. The kit of claim 36, wherein said allelic variant comprises an A at position 43,157,041 of the KCNS1 sequence or said allelic variant comprises a G at position 43,155,431, A at position 43,157,041, and C at position 43,160,569 of the KCNS1 sequence.

40. A kit for predicting pain sensitivity, diagnosing the risk of developing acute or chronic pain, or diagnosing the risk of developing an BH4-related disorder in a mammalian subject, said kit comprising:
(a) an agent for increasing cyclic AMP levels in a cell, LPS, or an inflammatory cytokine;
(b) a first primer for hybridization to a GTP cyclohydrolase (GCH1) mRNA sequence; and
(c) instructions for use.

41. The kit of claim 40, wherein said agent is an adenyl cyclase activator or a phosphodiesterase inhibitor.

42. The kit of claim 41, wherein said agent is forskolin.

43. The kit of claim 40, further comprising a second primer, wherein said first and second primers are capable of being used to amplify at least a portion of said GCH1 mRNA sequence.

44. A kit for predicting pain sensitivity, diagnosing the risk of developing acute or chronic pain, or diagnosing the risk of developing an BH4-related disorder in a mammalian subject, said kit comprising:
(a) an agent for increasing cyclic AMP levels in a cell, LPS, or an inflammatory cytokine;
(b) an antibody specific for GTP cyclohydrolase (GCH1); and
(c) instructions for use.

45. The kit of claim 44, wherein said agent is an adenyl cyclase activator or a phosphodiesterase inhibitor.

46. The kit of claim 45, wherein said agent is forskolin.
